(19) United States
(12) Patent Application Publication
(10) Pub. No.: US 2024/0229138 A9
(43) Pub. Date: Jul. 11, 2024

(54) FLUIDIC DEVICE AND METHODS FOR CHARACTERIZATION OF AN INFECTION OR OTHER CONDITION

(71) Applicant: Inflammatix, Inc., Sunnyvale, CA (US)

(72) Inventors: Timothy Elisha Sweeney, Sunnyvale, CA (US); João Manuel de Oliveira Garcia da Fonseca, Sunnyvale, CA (US); Paul Michael Fleming, Sunnyvale, CA (US); Anna Claire Passernig, Austin, TX (US); Kevin Hu, Palo Alto, CA (US); Ragheb Mohamad Fawaz El Khaja, Oakland, CA (US)

(21) Appl. No.: 18/493,324

(22) Filed: Oct. 24, 2023

Related U.S. Application Data
(60) Provisional application No. 63/418,863, filed on Oct. 24, 2022.

(51) Int. Cl. C12Q 1/6883; B01L 3/00; C12Q 1/6844

(52) U.S. Cl. CPC C12Q 1/6883; B01L 3/502707; B01L 3/502738; C12Q 1/6844; B01L 2200/0642; B01L 2200/16; B01L 2400/0638; C12Q 2600/158

(57) ABSTRACT

A fluidic device, instrument, and method for processing a biological sample is disclosed herein. An example of the fluidic device includes structures (e.g., sample holder(s), reaction chamber(s), reagent storage zone(s), metering compartment(s), reaction well(s), detection well(s), channel(s), etc.) for efficiently processing sample components and providing expression levels of various biomarker components for characterization of a sample.

### CROSS-REFERENCE TO RELATED APPLICATIONS

This application claims the benefit of U.S. Provisional Application Ser. No. 63/418,863, filed on 24 Oct. 2022, which is incorporated in its entirety by this reference.

### TECHNICAL FIELD

This invention relates generally to the molecular diagnostics field, and more specifically to an improved fluidic device for characterizing a sample.

### BACKGROUND

Measuring gene expression can be useful in diagnostic and prognostic applications. For example, measuring the expression of genes in the host response pathway may be useful in the characterization (e.g., diagnosis, prognosis, etc.) of a disease (e.g., acute disease, chronic disease), disorder, or condition. The disease, disorder, or condition can be associated with changes in a subject's immune system that can be analyzed to inform on the disease, disorder, or condition. Infections are one such example of a disease, disorder, or condition that may be associated with a change in a subject's immune system. Infections are usually caused by microscopic pathogens, such as viruses, bacteria, and fungi. While some infections are mild and can resolve without treatment, some infections are severe and require treatment or intervention. Viral infections can usually be treated with antiviral therapies while subjects with bacterial infections can be treated with antibiotics. Fungal infections can be treated with antifungals. Knowing the type of infection, therefore, can be important to inform on the type of treatment to use. Some subjects may develop severe disease and require medical care at, for example, a hospital. Identifying a subject that has severe disease and requires more intensive medical care is important so that the subject receives timely medical attention. Timely and accurate diagnosis of a disease, disorder, or condition can be extremely important for the treatment and prognosis of a subject, for example in the case of acute infection.

There remains a need for improved devices and methods which can assist clinicians in accurately and timely diagnosing and treating a patient with a disease, disorder, or condition such as an infection.

### SUMMARY OF THE INVENTION

Currently, systems and methods for processing a biological sample, at the point of care (PoC), in order to accurately analyze biomarker expression levels of a number of biomarkers with simultaneous reactions (e.g., isothermal reactions, such as loop-mediated isothermal amplification (LAMP) reactions), and with minimal or no user sample pre-processing involvement, are severely limited. As such, embodiments, variations, and examples of a system of the present disclosure cover a fluidic device and an instrument that operate together to determine expression levels of biomarkers from a biological sample of a subject. The system can be used as a rapid, point-of-care diagnostic tool that receives an unprocessed sample directly from the patient, and rapidly returns results indicative of biomarker expression for a large number of relevant biomarkers. In examples, the unprocessed sample is a sample that is received from a subject without any manipulation (e.g., without combination with reagents, without washing, without lysis, without pre-amplification, etc.) prior to delivery into the system.

In examples, embodiments of the system and methods implemented by the system can simultaneously achieve: reception of an unprocessed sample at the point-of-care, detection of expression levels of a large number of biomarkers with simultaneous amplification reactions performed in parallel and with multiplexed detection capability, and returning of results informative of a subject's condition (e.g., prognosis/diagnosis of an infection type, as well as distinguishing infection from sterile inflammation) in an extremely rapid manner. In one exemplary use case, the system can be structured as a point-of-care portable cartridge for receiving an unprocessed whole blood sample from a subject, processing the whole blood sample within microfluidic structures with performance of simultaneous isothermal amplification reactions having high selectivity for enabling detection of expression levels of a large panel of biomarkers (e.g., greater than 20 biomarkers), and providing results indicative of a type and/or severity of infection present in the subject (from analysis of host-sourced biomarkers without analysis of pathogen-sourced biomarkers), within a short period of time (e.g., within 5 minutes).

In more detail, embodiments, variations, and examples of the invention(s) covered can be structured to detect a plurality of genes, with exemplary performance for detection of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, or more than 200 unique genes or biomarker genes.

Additionally, embodiments, variations, and examples of the invention(s) covered can be structured to provide environments for high-throughput reaction performance, signal detection, and diagnostic output within a short duration of time, with exemplary performance for completion of analysis within 20 minutes, 21 minutes, 22 minutes, 23 minutes, 24 minutes, 25 minutes, 26 minutes, 27 minutes, 28 minutes, 29 minutes, 30 minutes, 31 minutes, 32 minutes, 33 minutes, 34 minutes, 35 minutes, 36 minutes, 37 minutes, 38 minutes, 39 minutes, 40 minutes, 41 minutes, 42 minutes, 43 minutes, 44 minutes, 45 minutes, 46 minutes, 47 minutes, 48 minutes, 49 minutes, 50 minutes, 51 minutes, 52 minutes, 53 minutes, 54 minutes, 55 minutes, 56 minutes, 57 minutes, 58 minutes, 59 minutes, or about 60 minutes, or any time therebetween.

Additionally, embodiments, variations, and examples of the invention(s) covered can be structured to support isothermal reactions (e.g., LAMP) and/or other amplification reactions, such as polymerase chain reaction (PCR) amplification, recombinase polymerase amplification (RPA), ligase chain reaction, branched DNA amplification, nucleic acid sequence-based amplification (NASBA), strand displacement assay (SDA), transcription-mediated amplification, rolling circle amplification, helicase-dependent amplification (HDA), single primer isothermal amplification (SPIA), nicking and extension amplification reaction (NEAR), or transcription mediated assay (TMA).

In an embodiment, a method for evaluating a subject can include: loading a biological sample of the subject into a fluidic device, wherein the biological sample comprises a volume of whole blood; performing a set of processes within the fluidic device; and measuring an expression level of a composite biomarker derived from a set of subcomponents present in the biological sample, using the fluidic device, wherein the subject is identified as having or not having an infection based on the expression level of the composite biomarker, and wherein the method takes no more than a period of time (e.g., 35 minutes).

In particular, embodiments, variations, and examples of the method achieve unprecedented performance in runtime, sample volume required to return characterizations of a biological sample, and other performance aspects described, based upon the following factors and other factors described in the Detailed Description:

Embodiments, variations, and examples of the method omit performance of deoxyribonucleic acid (DNA) degradation involving a Deoxyribonuclease (DNase), with omission of amplification of genomic DNA. As such, the method can be performed with elimination of steps involving DNase use, as attributed to primer design factors described herein. However, variations of the method can include performance of degradation involving DNase.

Embodiments, variations, and examples of the method include combining the biological sample with an intracellular RNA stabilization solution (e.g., PAXgene™) for biological samples including intracellular ribonucleic acids (RNAs), given that lysis has already occurred prior to introduction to the fluidic device. However, variations of the method can include performance of lysis on-cartridge (e.g., within the fluidic device).

Embodiments, variations, and examples of the method implement a significant reduction in number of wash steps, by executing a LAMP assay with tolerance to residual inhibitors. In examples, the number of wash steps can be less than 10 wash steps, less than 9 wash steps, less than 8 wash steps, less than 7 wash steps, less than 6 wash steps, less than 6 wash steps, less than 5 wash steps, less than 4 wash steps, less than 3 wash steps, or less than 2 wash steps.

Embodiments, variations, and examples of the method apply active drying (e.g., forced convection drying) of biological reagents stored on the fluidic device, in order to ensure stability at room temperature (or another suitable temperature), and wherein said biological reagents are used for the set of processes, such that processing the biological sample with dried on-cartridge reagents can be performed extremely efficiently.

Embodiments, variations, and examples of the method involve performing fluid agitation (e.g., pneumatic-driven fluid agitation) and homogenization of functionalized particles, with bubble mixing, within the fluidic device, thereby producing rapid binding of RNAs of the biological sample to capture molecules within the fluidic device and significantly reducing runtime.

Embodiments, variations, and examples of the method involve parallelization of sample processing steps, including but not limited to sample metering, reagent rehydration, heating of buffers, dispensing wet reagents, and other sample processing steps.

Embodiments, variations, and examples of the method can be executed with significant improvements in the required blood volume needed for processing, due to selection of appropriately abundanced targets of the sample that are analyzed for returning characterizations. In examples, the volume of a biological sample (e.g., blood sample combined with an RNA stabilization component) processed using the fluidic device is less than 1 mL, and the fluidic device is configured to perform (e.g., optimized for) sample processing and characterization based upon a first set of informative biomarker components (i.e., informative genes), a second set of housekeeping biomarker components (e.g., housekeeping genes), and a third set of control components (e.g., control genes). Detection and analysis of the first set of informative biomarker components (i.e., informative genes) provides information regarding the condition of the subject (where exemplary conditions are described herein), the second set of housekeeping biomarker components (e.g., housekeeping genes) provides control from subject-to-subject, and the third set of control biomarker components (e.g., control genes) are indicative of proper functioning of the fluidic device (e.g. in relation to cartridge-to-cartridge consistency and reproducibility of analyses), with respect to proper sample preparation and proper amplification, respectively. The third set of control biomarker components is also used to normalize/provide corrections for characterizations (e.g., abundance characterizations) of other biomarker components.

The selection of the first set of informative biomarkers, the second set of housekeeping biomarkers, and the third set of control biomarkers is based upon an assessment of biomarker targets that amplify well, using the fluidic device. Upon processing the biological sample and analyzing signals related to the first set of informative biomarkers, the second set of housekeeping biomarkers, and the third set of control biomarkers, the system and method are structured to return a characterization of the biological sample based upon a composite biomarker.

According to a specific example, the first set of informative biomarkers includes the following genes: ANKRD22, ARG1, BATF, C3AR1, CD163, CEACAM1, CLEC5A, CTSL1, DEFA4, HERC5, HLA-DMB, IFI27, IFI44, IFI44L, IL18R1, IL1R2, ISG15, JUPv9, KCNJ2, LY86, OASL, OLFM4, PSMB9, RSAD2_PT4, S100A12_PT1, TDRD9_PT3, TGFBI, XAF1_PT4, and ZDHHC19.

According to a specific example, the second set of housekeeping biomarkers includes the following genes: KPNA6, RREB1, and YWHAB.

According to a specific example, the third set of control biomarkers includes RNA controls (e.g., ERCC17, ERCC59, additional or alternative control biomarkers, etc.).

Monitoring of signals associated with the first set of informative biomarkers, the second set of housekeeping biomarkers, and the third set of control biomarkers at the fluidic device (e.g., on-cartridge) can be performed in real-time during amplification and readout at the fluidic device, in order to provide real-time or near-real-time assessments of proper fluidic device function, proper sample preparation, and/or proper amplification.

Alternatively, detection of signals associated with the first set of informative biomarkers, the second set of housekeeping biomarkers, and the third set of control biomarkers at the fluidic device (e.g., on-cartridge) can be performed after processing of the sample (e.g., after performance of the entire assay, such as a LAMP assay), in order to return indications of assessments of proper fluidic device function, proper sample preparation, and/or proper amplification.

Embodiments, variations, and examples of a system provide enhanced sample handling with the fluidic device, by incorporating features that control fluid flow from a collection tube or other container upon coupling the collection tube/container to the fluidic device. Such features provide increased resistance to opening valves, in a highly controlled manner and in response to a user action (e.g., at the point-of-care), such that the method can be executed in a reliable manner and with minimal user training. Such features can also include features that resist fluid flow (or prevent fluid flow) below a threshold and/or tunable pressure (e.g., cracking pressure).

Embodiments, variations, and examples of the inventions are configured to generate and/or provide notifications (e.g., to an operator of a fluidic device described, to an operator of an instrument described, etc.) regarding quality of sample drawing (e.g., if a blood draw was suitable or unsuitable, if sample metering at the fluidic device was performed correctly, etc.), for instance, using a display and/or connected device. Embodiments, variations, and examples of the inventions are configured to generate and/or provide notifications (e.g., to an operator of a fluidic device described, to an operator of an instrument described, etc.) regarding any prior use of the fluidic device (e.g., for cartridges or other disposable components that should not be used multiple times). Identification that a fluidic device has been previously used can be determined upon scanning a barcode associated with the fluidic device (e.g., barcode used to label a cartridge), where scanning the barcode can indicate a history of usage of the fluidic device. Scanning components of the instrument described below can include scanning elements (e.g., optical scanning, near-field communication scanning, radiofrequency identification scanning, etc.) for determining usage history of a fluidic device unit. Identification that a fluidic device has been previously used can be determined upon scanning a barcode associated with the fluidic device (e.g., barcode used to label a cartridge), where scanning the barcode can indicate a history of usage of the fluidic device. Identification that a fluidic device has been previously used can additionally or alternatively be determined upon sensing presence of fluid (or evidence of fluid) within components (e.g., chambers, channels, wells, etc.) of the fluidic device, where sensing presence of fluid can be performed using one or more capacitive or optical sensors. The one or more capacitive/optical sensors can be positioned at the fluidic device and/or instrument described in further detail below.

Additional aspects and advantages of the present disclosure will become readily apparent to those skilled in this art from the following detailed description, wherein only illustrative embodiments of the present disclosure are shown and described. The present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference in their entireties for all purposes and to the same extent as if each individual publication, patent, or patent application was specifically and individually indicated to be incorporated by reference.

Furthermore, where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

### BRIEF DESCRIPTION OF THE FIGURES

FIG. 1 depicts a schematic of a system, including a fluidic device and an instrument, for characterization of a condition of a subject.

FIG. 2A depicts a first side of a fluidic device.

FIG. 2B depicts a second side of a fluidic device.

FIG. 3 depicts an instrument for receiving a fluidic device.

FIG. 4 depicts a portion of a fluidic device.

FIGS. 5A-5C depict aspects of a valve of an exemplary fluidic device.

FIG. 6 depicts a flowchart of an embodiment of a method using a fluidic device and instrument.

### DETAILED DESCRIPTION

While exemplary embodiments of the present disclosure will be shown and described herein through drawings and detailed description, it will be understood that such embodiments are provided by way of example only. Numerous variations, changes, and substitutions should be obvious to one skilled in the art without departing from the present disclosure. It should be understood that various alternatives to the embodiments of the invention described herein may be employed in practicing the invention.

### 1. General Overview and Benefits

Provided herein are systems, devices, instruments, compositions, and methods that can be employed for diagnosis and/or prognosis of a number of diseases, disorders, and conditions, particularly where differences in gene expression can be detected. The genes that are differentially expressed may be associated with a host immune response or immune system. The genes that are differentially expressed may be considered biomarkers for the disease, disorder, or condition. Such genes can thus operate as subcomponents of a sample being analyzed, where values of signals corresponding to the subcomponents can be used to generate a composite biomarker for characterizing a condition (e.g., bacterial infection, viral infection, sterile inflammation, state of severity of a condition, etc.) represented in the biological sample.

The systems, devices, instruments, compositions, and methods provided herein can be used for rapid, point-of-care diagnostic testing to enable faster times to diagnosis and/or treatment of a subject.

In the case of infections, an infectious pathogen such as, but not limited to, a virus, bacteria, fungi, protozoa, and worm, may activate a host's immune system. Immune effector cells, such as B cells, T cells, natural killer cells, neutrophils, eosinophils, basophils, mast cells, monocytes, macrophages, and dendritic cells, can respond to the infectious pathogens with a variety of cellular events, including increased or decreased expression of certain genes, or biomarkers. Changes in the expression levels of host response biomarkers can be detected and compared to, e.g., a subject that does not have the infection, to diagnose and/or determine the prognosis of a subject.

The inventions further provide benefits and advantages over existing systems and methods, and achieve unprecedented performance in runtime, sample volume required to return characterizations of a biological sample, sample processing through fluidic device design, and other performance aspects described, based upon the following factors and other factors:

Implementation of the invention(s): omit performance of deoxyribonucleic acid (DNA) degradation involving a Deoxyribonuclease (DNase), with omission of amplification of genomic DNA; combine a biological sample with an intracellular RNA stabilization solution (e.g., PAXgene™) for biological samples including intracellular ribonucleic acids (RNAs), given that lysis has already occurred prior to introduction to the fluidic device; implement a significant reduction in number of wash steps, by executing a LAMP assay with tolerance to residual inhibitors; apply active drying (e.g., forced convection drying) of biological reagents stored on the fluidic device to ensure stability at room temperature, wherein the biological reagents are used for the set of processes within the fluidic device, to achieve significant reductions in sample processing time; perform fluid agitation (e.g., pneumatic-driven fluid agitation) and homogenization of functionalized particles, with bubble mixing, within the fluidic device, thereby producing rapid binding of RNAs of the biological sample to capture molecules within the fluidic device and significantly reducing runtime; involve parallelization of sample processing steps, including but not limited to sample metering, reagent rehydration, heating of buffers, dispensing wet reagents, and other sample processing steps; require less blood volume needed for processing, due to selection of appropriately abundanced targets of the sample that are analyzed for returning characterizations; and provide enhanced sample handling with the fluidic device, by incorporating features that control fluid flow from a collection tube or other container upon coupling the collection tube/container to the fluidic device.

### 1.1 Definitions

The terminology used herein is for the purpose of describing particular cases only and is not intended to be limiting.

As used herein, unless otherwise indicated, the terms “a”, “an” and “the” are intended to include the plural forms as well as the single forms, unless the context clearly indicates otherwise.

The terms “comprise”, “comprising”, “contain,” “containing,” “including”, “includes”, “having”, “has”, “with”, or variants thereof as used in either the present disclosure and/or in the claims, are intended to be inclusive in a manner similar to the term “comprising.”

The term “prognosis” refers to the likely outcome or course of a disease or condition. In some cases, it may refer to the likelihood of recovery from a disease or condition or may refer to the likelihood of adverse outcomes, such as death.

The term “diagnosis” refers to determining the occurrence of a disease and/or the likelihood of having the disease.

The term “treatment” refers to ameliorating or avoiding the effects of a disease, including reducing a sign or symptom of the disease.

The term “about” means within an acceptable error range for the particular value as determined by one of ordinary skill in the art, which will depend in part on how the value is measured or determined, e.g., the limitations of the measurement system. For example, “about” can mean 10% greater than or less than the stated value. In another example, “about” can mean within 1 or more than 1 standard deviation, per the practice in the given value. Where particular values are described in the application and claims, unless otherwise stated the term “about” should be assumed to mean an acceptable error range for the particular value.

When an element is described as being “coupled,” “connected,” to, or “on,” another element, it can be directly coupled or connected to, or on, the other element, or intervening elements may also be present.

The terms “first,” “second,” “third”, etc. may be used herein to describe various elements which should not be limited by these terms. Thus, a “first” element could be termed a “second” element without departing from the teachings of the present disclosure.

A “biological sample” means a biological specimen, a cell, tissue, organ, fluid, or a biological material from a biological organism, such as bacteria, fungi, viruses, plants, animals, or humans.

The term “subject,” refers to a biological organism, such as bacteria, fungi, viruses, plants, animals, or humans.

A “nucleic acid” refers to a naturally occurring or synthetic oligonucleotide or polynucleotide, such as DNA, RNA, or DNA-RNA hybrid, single-stranded or double-stranded, sense or antisense, A nucleic acid may include, without limitation, DNA, RNA, cDNA, mRNA, tRNA, rRNA, gRNA, microRNA, genomic DNA or any combination thereof

The terms “primer,” or “oligonucleotide” refers to a single-stranded nucleic acid molecule of a defined sequence that can hybridize to a second nucleic acid molecule that contains a complementary sequence. Probes, primers, and oligonucleotides may be labeled with detectable groups, either radioactively, fluorescently, or non-radioactively, by other methods well-known to those skilled in the art. Double strand DNA (dsDNA) binding dyes may be used to detect dsDNA. A “primer” may be configured to be extended by a polymerase.

The terms “reverse transcribe,” or, “reverse transcription,” refers to the generation of a complementary DNA from a RNA, such as a messenger RNA (mRNA).

The term “isothermal amplification” refers to a process of enzymatically copying a nucleic acid molecule at a constant temperature to generate a population of nucleic acid molecules with the same sequence as the parental one.

The term “loop-mediated isothermal amplification (LAMP)” refers to an isothermal nucleic acid amplification process using pairs of primers and a polymerase with high strand displacement activity.

Any aspect or embodiment described herein can be combined with any other aspect or embodiment as disclosed herein.

### 2. System

An embodiment of a system 10 includes a fluidic device 100 and an instrument 300 that operate together to determine expression levels of subcomponents (e.g., biomarkers) from a biological sample of a subject, in order to generate a composite biomarker that can be used to characterize the biological sample. The system can be used as a rapid, point-of-care diagnostic to inform on a subject's status with respect to a disease, disorder, or condition. Embodiments, variations, and examples of the fluidic device 100 of the system 10 provided herein can be used for a rapid point-of-care transcriptomics test that measures expression levels of one or a plurality of biomarker genes for a disease, disorder, or condition. Genes refer to genomic sequences (e.g., deoxyribonucleic acid, DNA), contiguous or noncontiguous, that may encode for a product such as a ribonucleic acid (RNA) or protein. Some RNA function as messenger RNA, or mRNA, which are ultimately translated into proteins while some RNA function as non-coding RNA (e.g., transfer RNAs, ribosomal RNAs, microRNAs, siRNAs, piRNAs, snoRNAs, snRNAs, exRNAs, scaRNAs, and long ncRNAs) and may instead perform regulatory functions. Embodiments, variations, and examples of the fluidic device 100 can be used to determine the expression level of a composite biomarker gene derived from expression levels of one or more subcomponents (e.g., biomarkers) represented in the sample, by measuring the expression level of RNA and/or other nucleic acids corresponding to the biomarker gene. In some embodiments, the biomarker gene may be related to an immune response. In some embodiments, the expression levels of a plurality of biomarker genes are measured by the fluidic device 100.

Embodiments, variations, and examples of the fluidic device 100 can execute methods involving: receiving a biological sample obtained from a subject (also referred to interchangeably herein as a “patient”). After the biological sample is loaded into the fluidic device 100, the biological sample can be subjected to one or more reactions of a set of processes within the fluidic device 100 to determine expression levels of different subcomponents, which can be used to generate a composite biomarker. A method step can include: performing a nucleic acid purification operation to yield one or more purified nucleic acids from the biological sample. Where both the analysis of DNA and RNA are desired, the purified nucleic acids may comprise both purified DNA and RNA. In some embodiments, the purified nucleic acid can comprise both purified DNA and RNA but only one type of nucleic acid is subsequently analyzed. Where the measurement of RNA expression level of a biomarker is desired, the fluidic device can 100, in some embodiments, extract RNA from the biological sample without extracting the DNA, and/or can omit use of a DNase. In some embodiments, the RNA comprises messenger RNA, or mRNA. In some embodiments, the RNA comprises miRNA.

After nucleic acid extraction, embodiments, variations, and examples of the fluidic device 100 can then determine the expression levels of one or more subcomponents of a set of subcomponent (e.g., biomarker genes), in a highly-multiplexed manner, by measuring the levels of corresponding mRNA or other sample targets. In some embodiments, measuring the levels of mRNA can be accomplished by an amplification reaction performed within the fluidic device 100. In some embodiments, measuring the levels of mRNA can be accomplished by another detection method within the fluidic device, such as a CRISPR-Cas detection reaction. To perform an amplification reaction, the fluidic device may contain a set of nucleic acid primers capable of amplifying a nucleic acid, such as an RNA, corresponding to each subcomponent (e.g., biomarker gene). The specific sequence(s) of the primers and number of primers within the primer set can be tailored to the type of amplification reaction to be performed. For example, the amplification could comprise an isothermal reaction. In some cases, the isothermal reaction is a loop-mediated isothermal amplification reaction (LAMP).

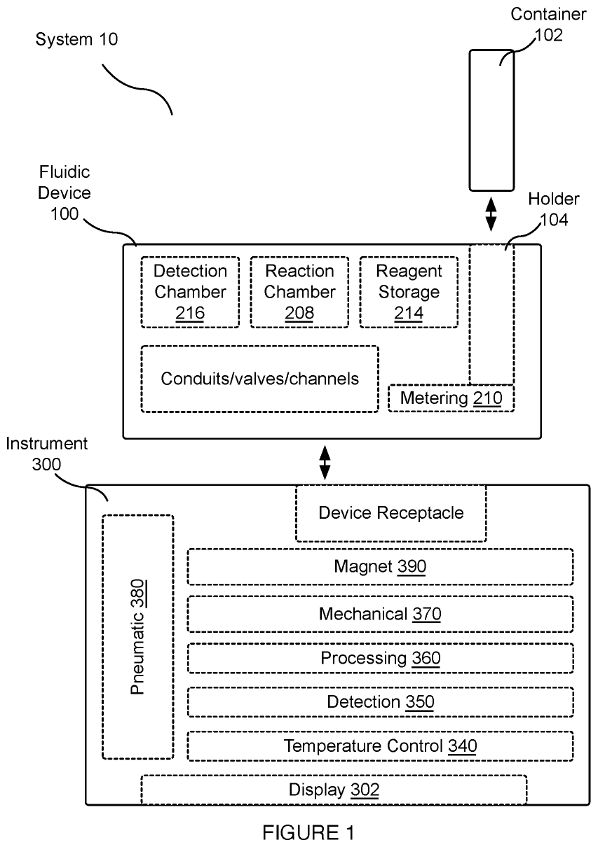

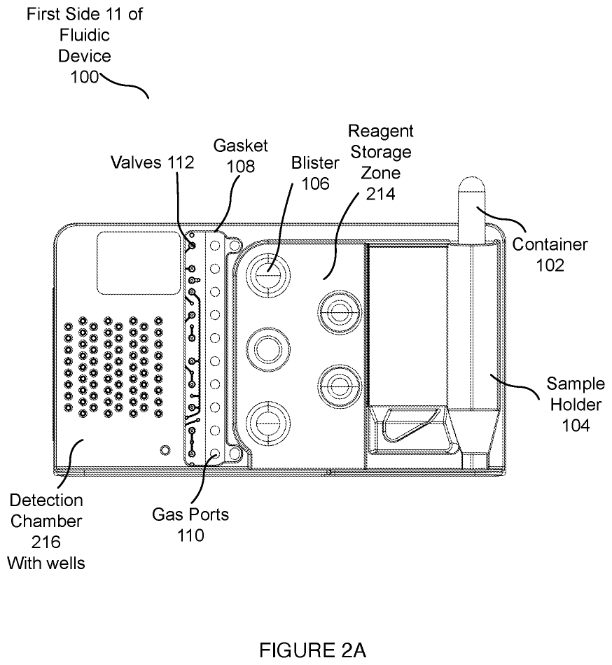

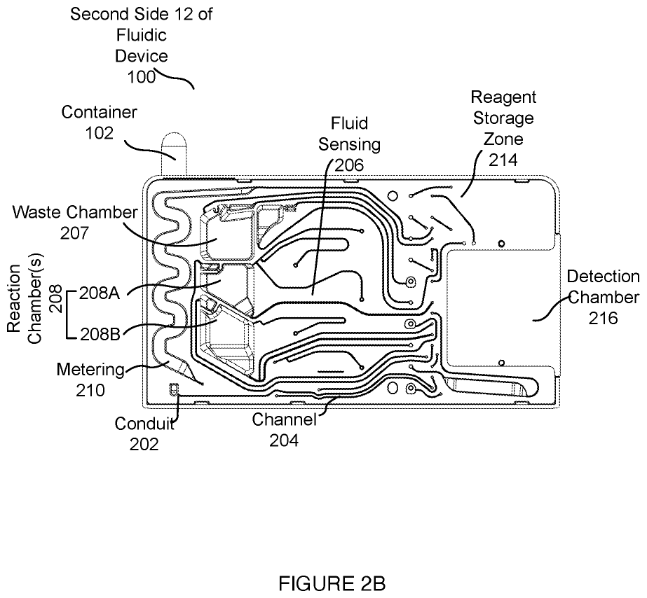

As shown in FIGS. 1, 2A, and 2B, an example of the fluidic device 100 can include show a first side 11 and a second side 12. The fluidic device 100 can include reagents and reaction compartments to perform nucleic acid extraction, nucleic acid amplification, and nucleic acid detection.

### 2.1 System—General

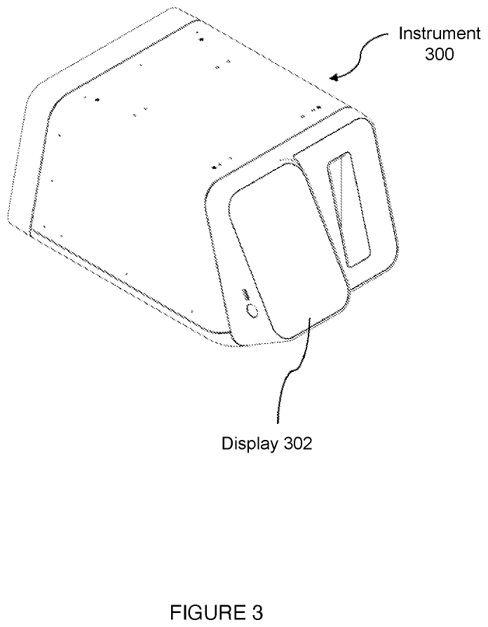

In some embodiments, the fluidic device 100 can have the form of a cartridge that can be inserted into an instrument 300 shown in FIG. 3 and described in more detail in Section 2.4 below. In some embodiments, the fluidic device 100 can be a single use device. In some embodiments, the biological sample, within a container (e.g., collection tube), is loaded into the fluidic device 100 which is then inserted into the instrument so that the biological sample is processed by the fluidic device and/or the instrument 300. In some embodiments, the instrument 300 can contain computer software and electronics for programming and executing the methods disclosed herein. In some embodiments, the fluidic device 100 can contain computer software and electronics for programming and executing the methods disclosed herein.

In some embodiments, the instrument 300 can include a display panel 302, as shown in FIG. 3. The display panel 302 can be configured to display information related to the processing of the biological sample and/or diagnosis of a disease, disorder, or condition based on the information obtained from the biological sample. For example, the display panel may allow a user to choose a type of program used for processing the biological sample. The display panel 302 can include architecture with input controls for allow a user to input other information as needed, for example a sample ID or other patient clinical or demographic information. In some embodiments, the display panel 302 can include architecture for displaying the result after the biological sample is processed. In some embodiments, the display panel 302 can include architecture for displaying the diagnosis result based on information obtained from the biological sample being processed. In some embodiments, the display panel 302 can include a touchscreen and a control menu (e.g., as input subsystems) allowing a user to make configurations. In some embodiments, the instrument 300 can be configured to communicate wirelessly with another device and/or software application, such as a cell phone and/or a mobile application. In some embodiments, the instrument 300 may be configured to be controllable through a mobile application and/or a cell phone.

### 2.2 System—Sample Reception

In embodiments, the fluidic device 100 can include a sample holder 104 that is configured to hold the biological sample. In embodiments, the biological sample can be loaded into an internal sample container that is built into the fluidic device 100. In embodiments, the biological sample may be contained in an external sample container 102 (e.g., collection tube) that is inserted into and held inside the sample holder 104. In some embodiments, the sample container 102 can be removable from the sample holder 104. In some embodiments, the sample container 102 may be a vial, a tube, a bottle, or a cup. In some embodiments, the sample container 102 can be a vacuum blood collection tube. The sample container 102 can contain (e.g., be pre-packaged, be combined with the biological sample prior to coupling the sample container 102 to the fluidic device 100) one or more additives.

In examples, additives can include one or more of: a clot activator, a thrombin-based clot activator, a clot activator and gel for serum separation, a lithium heparin and gel for plasma separation, a thrombin-based clot activator with gel for serum separation, EDTA, K2EDTA, sodium heparin, lithium heparin, potassium oxalate/sodium fluoride, sodium fluoride/Na2 EDTA, sodium fluoride, sodium polyanethol sulfonate, acid citrate dextrose additives, K3EDTA, sodium citrate, and CTAD (citrate, theophylline, adenosine, dipyridamole). For example, the sample container may be a Vacutainer®, PAXgene® Blood Tube (e.g., RNA, DNA), Tempus Blood RNA Tube, or any other commercially available container. Where analysis of RNA is desired, the sample container can contain an RNA stabilization agent to prevent or minimize the degradation of RNA during storage and before the biological sample is processed. In some embodiments, the biological sample can be directly loaded into the sample holder 104 without a sample container 102. In some embodiments, the sample holder 104 can include an opening for directly loading the biological sample without using a sample container and a closable compartment for holding the biological sample.

In some embodiments, the biological sample is subject to processing before being loaded into the sample container 102. In some embodiments, the biological sample is not subject to processing before being loaded into the sample container 102. In some embodiments, the biological sample is subject to processing before being loaded into the sample holder 104. In some embodiments, the biological sample is not subject to processing before being loaded into the sample holder 104. In some embodiments, the biological sample is subject to processing after being loaded into the sample container but before the sample container containing the biological sample is loaded into the fluidic device 100. In some embodiments, the biological sample is not subject to processing after being loaded into the sample container but before the sample container containing the biological sample is loaded into the fluidic device 100. In some embodiments, the biological sample is not subject to manipulation or tampering by a user before or after the biological sample is loaded into the sample container 102. In some embodiments, the biological sample may be a biological specimen obtained from a subject. In some embodiments, the biological sample may be in solid form. In some embodiments, the biological sample may be in liquid form. In some embodiments, the biological sample may be converted from a solid form to a liquid form before being loading into the fluidic device 100. In some embodiments, the biological sample may comprise whole blood, a buffy coat, plasma, serum, saliva, urine, tissue biopsy, peripheral blood mononucleated cell (PBMC), a band cell, a neutrophil, a monocyte, a T cell, a nasal swab, or a combination thereof.

In variations, the sample can include a blood sample that is combined with materials for stabilization of RNA (e.g., stabilization of intracellular RNA), wherein, in examples, the materials can include PAXgene® or other sample stabilization components.

In examples, the sample has a volume from 100 microliters to 1500 microliters; however, the sample can alternatively have a volume less than 200 microliters or greater than 1500 microliters. In examples, the sample has a volume of approximately 100 microliters, approximately 150 microliters, approximately 200 microliters, approximately 250 microliters, approximately 300 microliters, approximately 350 microliters, approximately 400 microliters, approximately 450 microliters, approximately 500 microliters, approximately 550 microliters, approximately 600 microliters, approximately 650 microliters, approximately 700 microliters, approximately 750 microliters, approximately 800 microliters, approximately 850 microliters, approximately 900 microliters, approximately 950 microliters, approximately 1000 microliters, approximately 1050 microliters, approximately 1100 microliters, approximately 1150 microliters, approximately 1200 microliters, or greater. Smaller volumes of sample (e.g., volumes less than 500 microliters) include a smaller amount of target biomarker material, and also a smaller amount of inhibitor material for assays, thereby allowing assays to be run on the fluidic device 100, even with smaller input volumes.

In examples, the amount of blood and PAXgene® in the sample can be configured to provide suitable sample stabilization and an input amount of target biological material. In examples, the amount of blood in the sample can be 2.5% by volume, 5% by volume, 7.5% by volume, 10% by volume, 12.5% by volume, 15% by volume, 17.5% by volume, 20% by volume, 22.5% by volume, 25% by volume, 27.5% by volume, 30% by volume, 32.5% by volume, 35% by volume, 37.5% by volume, 40% by volume, 42.5% by volume, 45% by volume, 47.5% by volume, 50% by volume, 52.5% by volume, 55% by volume, 57.5% by volume, 60% by volume, 62.5% by volume, 65% by volume, 67.5% by volume, 70% by volume, 72.5% by volume, 75% by volume, 77.5% by volume, 80% by volume, 82.5% by volume, 85% by volume, 87.5% by volume, 90% by volume, 92.5% by volume, 95% by volume, 97.5% by volume, 100% by volume, or an intermediate percentage by volume.

In a specific example, the ratio of blood:PAXgene® in the sample is 2.5:6.9, and the sample has an input volume (e.g., post-metering within the fluidic device) of 500 microliters, where the input volume (e.g., after metering with metering component 210 described below) is 500 microliters. However, other suitable volumes can be controllably metered (e.g., caused to flow until a sensor is tripped) into fluidic channels and chambers of the fluidic device 100, as described.

### 2.3 System—Sample Processing

In embodiments, the fluidic device 100 includes a reagent storage zone 214, as shown in FIGS. 1, 2A, and 2B. The reagents in the reagent storage zone 214 can include reagents for performing nucleic acid extraction. In some embodiments, the reagent storage zone 214 can include a plurality of compartments to store one or more types of reagents. In some embodiments, a compartment of the reagent storage zone 214 includes a blister 106 or other container (e.g., well) that can be accessed (e.g., through opening of a seal, through use of one or more valves, etc.), as shown in FIGS. 1 and 2A. In some embodiments, the blister 106 can contain a reagent for processing the biological sample. In some embodiments, the reagent storage zone 214 can include a plurality of blisters. Where there are a plurality of blisters in a fluidic device 100, each of the plurality of blisters can contain a reagent. The reagents in individual blisters may contain different reagents, but in some cases, two or more blisters may contain the same reagent. In some embodiments, a reagent stored in a blister may be in liquid form. In some embodiments, the reagent may flow out of the blister when the blister is compressed and ruptured by a mechanical member. In some embodiments, the mechanical member may be located in the instrument 300. In some embodiments, the mechanical member may be controllable to compress the blister 106 allowing the reagent to flow out of the blister.

In some embodiments, the fluidic device 100 can include a conduit 202 which functions to transfer the biological sample out of the sample container 102. In some embodiments, the conduit 202 can be coupled to the sample holder 104. In some embodiments, the conduit 202 can be in contact with the biological sample. In some embodiments, the conduit can include a fixture, such as a needle, which accesses the biological sample in the sample container. For example, when the sample container is inserted into the sample holder 104, the needle can align with and automatically pierce the sample container 102, allowing the biological sample to enter the conduit 202. In some embodiments, the biological sample can be drawn into the conduit 202 (e.g., through capillary force, through applied pressure, etc.) and transferred to another component of the fluidic device 100 through a channel, an example of which is shown as 204. Fluidic devices of the disclosure can have a plurality of channels which facilitate flow of fluid inside the fluidic device between different compartments. A fluid may include a biological sample, a reagent solution, a gas, water, air, or a combination thereof.

In some embodiments, a channel can couple two or more of the components of the fluidic device. In some embodiments, the fluidic device comprises a plurality of channels. Two or more of the plurality of channels may be in fluid communication. One or more of the plurality of channels may be closable. In some embodiments, a fluid cannot flow through a channel when the channel is closed. In some embodiments, one or more of the channels of the fluidic device may be closable by a valve. A valve that closes a channel may be referred to as an active valve. In some embodiments, the valve may be located in the interior of the channel. In some embodiments, the valve is located at a terminus of the channel. In some embodiments, a fluid may flow through the channel when the valve is open. In some embodiments, a fluid cannot flow through the channel when the valve is closed. In some embodiments, the valve is controllable. In some embodiments, the valve is controllable through mechanical activation (e.g., for normally-open valves, the valves can be transitioned to a closed state using one or more pins that provide a biasing force against the valves, thereby closing off their corresponding channels), where the instrument 300 described below includes mechanical actuators for displacing pins that contact and close respective valves of the fluidic device 100. In some embodiments, the valve is controllable through a pump or a pneumatic assembly. In some embodiments, the valve is controllable electronically. In some embodiments, the valve is operatively coupled to a pump or a pneumatic assembly.

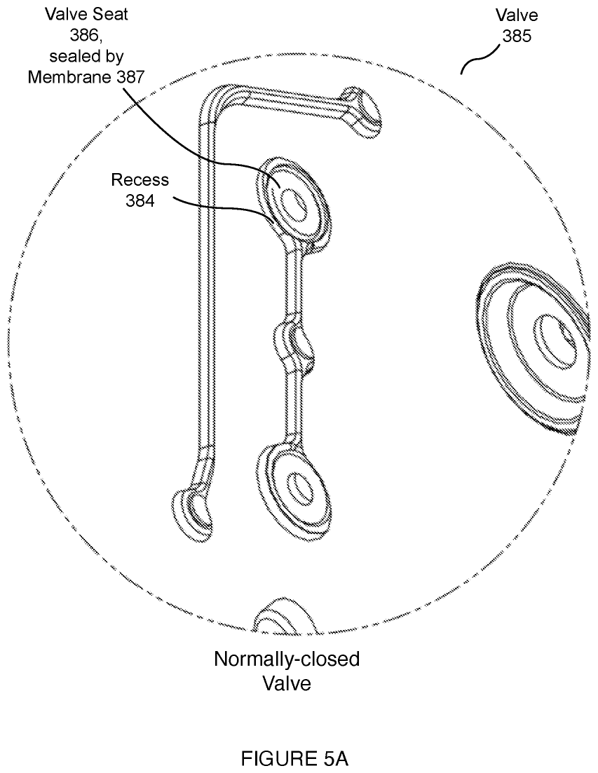

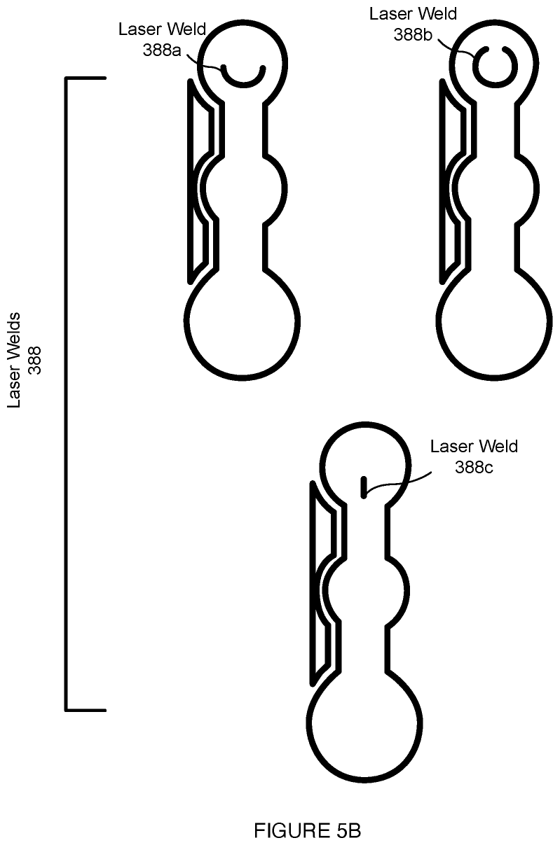

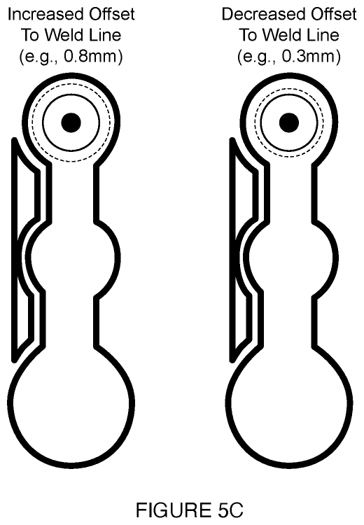

In the example shown in FIG. 5A, the fluidic device 100 can include a valve 385 provided in a normally-closed operation mode, where the valve 385 is configured to provide controlled (e.g., increased) resistance to opening the valve 385 when the sample container 102 interfaces with the fluidic device 100 at the device holder 104 by: providing the valve 385 with a valve seat 386 surrounded by a recess for a membrane 387 (e.g., an elastomeric membrane). The valve seat 386 can be a) flush with the membrane 387 or b) raised beyond the membrane 387 in order to provide greater resistance. As such, the normally-closed valve 385 can be positioned at an interface between the collection tube 102 and the fluidic device 100 (e.g., cartridge), in order to provide control over fluid delivery from the collection tube 102 into the fluidic device 100 (e.g., when a user docks the collection tube at the fluidic device 100/cartridge), such that processing the sample can be performed with greater control (e.g., upon inserting the fluidic device 100 into the instrument 300).

The valve 385 can further be structured to provide increased resistance to opening of the valve 385 when a collection tube 102 is inserted into holder 104 of the fluidic device 100 (e.g., upon interfacing the collection tube with the fluidic device), by laser welding (or otherwise coupling) the membrane 387 to the valve seat 386 in a manner that increases resistance to opening, such that opening the valve 385 occurs at a threshold cracking pressure.

In relation to welding/coupling the membrane 387 to the valve seat 386, the valve 385 can be structured with a weld 388 (e.g., laser weld, thermal bond, other bond, etc.) at one side of the valve seat, where the valve stays in the normally-closed operation mode until a cracking pressure is exceeded upon coupling the collection tube with the fluidic device 100. Welding the membrane 387 to the valve seat 386 itself substantially increases resistance, and the resistance can be tuned by the amount of welding, and the shape of the weld on the valve seat 386. As shown in FIG. 5B, the shape of the weld 388 can take the form of one or more radial welds 388a, 388b that radially span a portion of the valve seat (or span a portion of the perimeter of the valve seat). In examples, the radial weld can span approximately 5 degrees, 10 degrees, 15 degrees, 20 degrees, 25 degrees, 30 degrees, 35 degrees, 40 degrees, 45 degrees, 50 degrees, 55 degrees, 60 degrees, 65 degrees, 70 degrees, 75 degrees, 80 degrees, 85 degrees, 90 degrees, 95 degrees, 100 degrees, 105 degrees, 110 degrees, 115 degrees, 120 degrees, 125 degrees, 130 degrees, 135 degrees, 140 degrees, 145 degrees, 150 degrees, 155 degrees, 160 degrees, 165 degrees, 170 degrees, 175 degrees, 180 degrees, 185 degrees, 190 degrees, 195 degrees, 200 degrees, 205 degrees, 210 degrees, 215 degrees, 220 degrees, 225 degrees, 230 degrees, 235 degrees, 240 degrees, 245 degrees, 250 degrees, 255 degrees, 260 degrees, 265 degrees, 270 degrees, 275 degrees, 280 degrees, 285 degrees, 290 degrees, 295 degrees, 300 degrees, 305 degrees, 310 degrees, 315 degrees, 320 degrees, 325 degrees, 330 degrees, 335 degrees, 340 degrees, 345 degrees, 350 degrees, 355 degrees, 360 degrees or an intermediate number of degrees. Radial welds can be continuous, or intermittently positioned about the valve seat 386.

As shown in FIG. 5B, the shape of the weld 388 can take the form of one or more spot/line welds 388c, where the spot/line welds have a length of approximately 0.05 mm, 0.1 mm, 0.15 mm, 0.2 mm, 0.25 mm, 0.3 mm, 0.35 mm, 0.4 mm, 0.45 mm, 0.5 mm, 0.55 mm, 0.6 mm, 0.65 mm, 0.7 mm, 0.75 mm, 0.8 mm, 0.85 mm, 0.9 mm, 0.95 mm, 1 mm, or another suitable length. A specific example of the weld 388 includes a 0.2 mm line/spot weld, on the valve seat 386 on one side of the seat, which increases the fluidic resistance so that the valve stays closed until a threshold cracking pressure is exceeded.

In examples, the threshold cracking pressure can be approximately 5 kPa, 7.5 kPa, 10 kPa, 15 kPa, 20 kPa, 25 kPa, 30 kPa, 35 kPa, 40 kPa, 45 kPa, 50 kPa, 55 kPa, 60 kPa, 65 kPa, 70 kPa, 75 kPa, 80 kPa, 85 kPa, 90 kPa, 95 kPa, 100 kPa, an intermediate cracking pressure, or a greater cracking pressure. A specific example of the weld 388 provides a threshold cracking pressure of approximately 20 kPa.

The valve 385 can further be structured to provide increased resistance to opening the valve 385 with the collection tube 102 by providing the weld 388 with an offset to an opening into the valve (as shown in FIGURE C) less than 1.5 millimeters, less than 1 millimeter, less than 0.9 millimeters, less than 0.8 millimeters, less than 0.7 millimeters, less than 0.6 millimeters, less than 0.5 millimeters, less than 0.4 millimeters, less than 0.3 millimeters, less than 0.2 millimeters, or less. In a specific example, the offset of the weld 388 position to the valve 385 (e.g., opening) was less than 0.3 mm.

In some embodiments, the channel can include a fluid sensing zone 206, as shown in FIGS. 2A and 2B. In some embodiments, the fluid sensing zone may detect the presence or absence of a fluid inside the channel. The presence or absence of a fluid inside the channel may inform on if one or more steps has been completed within the fluidic device 100. In some embodiments, the fluid sensing zone may include a fluid sensor which detects the presence or absence of a fluid. Fluid sensor components can further be used to enable detection of prior usage of the fluidic device 100, to prevent re-usage of disposable components. In some embodiments, the fluid sensor may be operatively coupled to a pump or a pneumatic assembly that controls the flow of a fluid inside the channel. In some embodiments, the fluid sensor can be electrically coupled with a controller and configured to send a signal, through the controller, to the pump or pneumatic assembly that controls the flow of a fluid inside the channel. The pump or pneumatic assembly may interface with the channel via a gas port. In some embodiments, the pump or pneumatic assembly may be configured to start and/or stop the flow of a liquid inside the channel based on the signal of the fluid sensor. For example, when a fluid sensor detects the absence of a fluid, the pump or pneumatic assembly may flow a fluid through the channel based on the feedback signal of the fluid sensor. When a fluid sensor detects the presence of a fluid, the pump or pneumatic assembly may stop the flow of a fluid through the channel based on the feedback signal of the fluid sensor. In some embodiments, the pump may be an air pump. In embodiments, the sensor can include a moisture sensor, an optical sensor, a temperature sensor, a capacitive sensor, or another type of sensor.

In some embodiments, the fluidic device can include a reaction chamber 208 downstream of the sample holder 104 and the reagent storage zone 214, wherein the reaction chamber 208 is fluidically to channels for conveying the biological sample and one or more reagents from the reagent storage zone. In some embodiments, the reaction chamber 208 can include one or more reaction compartments as needed to perform one or more reactions on the fluidic device 100. In some embodiments, the reaction chamber 208 may comprise at least 3 reaction compartments, for performing multiplexed assays in parallel. For example, the reaction chamber 107 may include reaction compartments 208A, 208B shown in FIG. 2B. In some embodiments, the reaction chamber 208 comprises 2 or fewer reaction compartments. The one or more reaction chambers may have any appropriate volume for carrying out a reaction as disclosed herein.

In examples, a reaction compartment may have a volume between about 0.5 mL and about 10 mL, between about 0.5 mL and about 9 mL, between about 0.5 mL and about 8 mL, between about 0.5 mL and about 7 mL, between about 0.5 mL and about 6 mL, between about 0.5 mL and about 5 mL, between about 0.5 mL and about 4 mL, between about 0.5 mL and about 3 mL, between about 0.5 mL and about 2 mL, between about 0.5 mL and about 1 mL, between about 1 mL and about 10 mL, between about 1 mL and about 9 mL, between about 1 mL and about 8 mL, between about 1 mL and about 7 mL, between about 1 mL and about 6 mL, between about 1 mL and about 5 mL, between about 1 mL and about 4 mL, between about 1 mL and about 3 mL, between about 1 mL and about 2 mL, between about 1.5 mL and about 10 mL, between about 1.5 mL and about 9 mL, between about 1.5 mL and about 8 mL, between about 1.5 mL and about 7 mL, between about 1.5 mL and about 6 mL, between about 1.5 mL and about 5 mL, between about 1.5 mL and about 4 mL, between about 1.5 mL and about 3 mL, between about 1.5 mL and about 2 mL, between about 2 mL and about 10 mL, between about 2 mL and about 9 mL, between about 2 mL and about 8 mL, between about 2 mL and about 7 mL, between about 2 mL and about 6 mL, between about 2 mL and about 5 mL, between about 2 mL and about 4 mL, between about 2 mL and about 3 mL, between about 2.5 mL and about 10 mL, between about 2.5 mL and about 9 mL, between about 2.5 mL and about 8 mL, between about 2.5 mL and about 7 mL, between about 2.5 mL and about 6 mL, between about 2.5 mL and about 5 mL, between about 2.5 mL and about 4 mL, between about 2.5 mL and about 3 mL, between about 3 mL and about 10 mL, between about 3 mL and about 9 mL, between about 3 mL and about 8 mL, between about 3 mL and about 7 mL, between about 3 mL and about 6 mL, between about 3 mL and about 5 mL, between about 3 mL and about 4 mL, between about 3.5 mL and about 10 mL, between about 3.5 mL and about 9 mL, between about 3.5 mL and about 8 mL, between about 3.5 mL and about 7 mL, between about 3.5 mL and about 6 mL, between about 3.5 mL and about 5 mL, between about 3.5 mL and about 4 mL, between about 4 mL and about 10 mL, between about 4 mL and about 9 mL, between about 4 mL and about 8 mL, between about 4 mL and about 7 mL, between about 4 mL and about 6 mL, between about 4 mL and about 5 mL, between about 4.5 mL and about 10 mL, between about 4.5 mL and about 9 mL, between about 4.5 mL and about 8 mL, between about 4.5 mL and about 7 mL, between about 4.5 mL and about 6 mL, between about 4.5 mL and about 5 mL, between about 5 mL and about 10 mL, between about 5 mL and about 9 mL, between about 5 mL and about 8 mL, between about 5 mL and about 7 mL, between about 5 mL and about 6 mL, between about 5.5 mL and about 10 mL, between about 5.5 mL and about 9 mL, between about 5.5 mL and about 8 mL, between about 5.5 mL and about 7 mL, between about 5.5 mL and about 6 mL, between about 6 mL and about 10 mL, between about 6 mL and about 9 mL, between about 6 mL and about 8 mL, between about 6 mL and about 7 mL, between about 6.5 mL and about 10 mL, between about 6.5 mL and about 9 mL, between about 6.5 mL and about 8 mL, between about 6.5 mL and about 7 mL, between about 7 mL and about 10 mL, between about 7 mL and about 9 mL, between about 7 mL and about 8 mL, between about 7.5 mL and about 10 mL, between about 7.5 mL and about 9 mL, between about 7.5 mL and about 8 mL, between about 8 mL and about 10 mL, between about 8 mL and about 9 mL, between about 8.5 mL and about 10 mL, between about 8.5 mL and about 9 mL, between about 9 mL and about 10 mL, and between about 9.5 mL and about 10 mL.

In some embodiments, a reaction compartment may have any volume between about 0.5 mL and about 10 mL, e.g., about 0.5 mL, 0.6 mL, 0.8 mL, 0.9 mL, 1.0 mL, 1.1 mL, 1.2 mL, 1.3 mL, 1.4 mL, 1.5 mL, 1.6 mL, 1.7 mL, 1.8 mL, 1.9 mL, 2.0 mL, 2.1 mL, 2.2 mL, 2.3 mL, 2.4 mL, 2.5 mL, 2.6 mL, 2.7 mL, 2.8 mL, 2.9 mL, 3.0 mL, 3.1 mL, 3.2 mL, 3.3 mL, 3.4 mL, 3.5 mL, 3.6 mL, 3.7 mL, 3.8 mL, 3.9 mL, 4.0 mL, 4.1 mL, 4.2 mL, 4.3 mL, 4.4 mL, 4.5 mL, 4.6 mL, 4.7 mL, 4.8 mL, 4.9 mL, 5.0 mL, 5.1 mL, 5.2 mL, 5.3 mL, 5.4 mL, 5.5 mL, 5.6 mL, 5.7 mL, 5.8 mL, 5.9 mL, 6.0 mL, 6.1 mL, 6.2 mL, 6.3 mL, 6.4 mL, 6.5 mL, 6.6 mL, 6.7 mL, 6.8 mL, 6.9 mL, 7.0 mL, 7.1 mL, 7.2 mL, 7.3 mL, 7.4 mL, 7.5 mL, 7.6 mL, 7.7 mL, 7.8 mL, 7.9 mL, 8.0 mL, 8.1 mL, 8.2 mL, 8.3 mL, 8.4 mL, 8.5 mL, 8.6 mL, 8.7 mL, 8.8 mL, 8.9 mL, 9.0 mL, 9.1 mL, 9.2 mL, 9.3 mL, 9.4 mL, 9.5 mL, 9.6 mL, 9.7 mL, 9.8 mL, 9.9 mL, or about 10.0 mL, or any amount therebetween.

In some embodiments, the biological sample may flow from the sample container 102 to a reaction chamber where the biological sample is processed. In some embodiments, a pump or pneumatic assembly may facilitate the transfer of the biological sample from the sample container 102 to the reaction chamber 208.

In some embodiments, a pre-set amount of the biological sample can be driven or otherwise flow (e.g., under capillary effect) into the reaction chamber 208. In some embodiments, the fluidic device can include a metering compartment 210, as shown in FIG. 2B, where the metering compartment 210 is structured to control metering of a volume of the biological sample into the fluidic device 100 for processing. In an example, the fluidic device 100 comprises one or more channels and valves coupling the sample holder 104 to the metering compartment 210, which has a predefined volume. The predefined volume of the biological sample in the metering compartment 210 can then be transferred to the reaction chamber 208. In some embodiments, the metering compartment can be coupled to a sensor, such as a liquid sensor. In some embodiments, the liquid sensor may be configured to turn off the pump or pneumatic assembly when the biological sample in the metering compartment reaches the predefined volume. In some embodiments, the sensor can include a moisture sensor, an optical sensor, a temperature sensor, a capacitive sensor, or another type of sensor.

In some embodiments, the predefined volume of the metering compartment 210 can be any volume between about 0 and about 10 ml, between about 0 and about 9 ml, between about 0 and about 8 ml, between about 0 and about 7 ml, between about 0 and about 6 ml, between about 0 and about 5 ml, between about 0 and about 4 ml, between about 0 and about 3 ml, between about 0 and about 2 ml, between about 0 and about 1 ml, between about 0 and about 0.5 ml, between about 0 and about 0.25 ml, between about 0 and about 0.125 ml, between about 1 and about 10 ml, between about 1 and about 9 ml, between about 1 and about 8 ml, between about 1 and about 7 ml, between about 1 and about 6 ml, between about 1 and about 5 ml, between about 1 and about 4 ml, between about 1 and about 3 ml, between about 1 and about 2 ml, between about 2 and about 10 ml, between about 2 and about 9 ml, between about 2 and about 8 ml, between about 2 and about 7 ml, between about 2 and about 6 ml, between about 2 and about 5 ml, between about 2 and about 4 ml, between about 2 and about 3 ml, between about 3 and about 10 ml, between about 3 and about 9 ml, between about 3 and about 8 ml, between about 3 and about 7 ml, between about 3 and about 6 ml, between about 3 and about 5 ml, between about 3 and about 4 ml, between about 4 and about 10 ml, between about 4 and about 9 ml, between about 4 and about 8 ml, between about 4 and about 7 ml, between about 4 and about 6 ml, between about 4 and about 5 ml, between about 5 and about 10 ml, between about 5 and about 9 ml, between about 5 and about 8 ml, between about 5 and about 7 ml, between about 5 and about 6 ml, between about 6 and about 10 ml, between about 6 and about 9 ml, between about 6 and about 8 ml, between about 6 and about 7 ml, between about 7 and about 10 ml, between about 7 and about 9 ml, between about 7 and about 8 ml, between about 8 and about 10 ml, between about 8 and about 9 ml, and between about 9 and about 10 ml.

In some embodiments, the pre-set volume of the metering compartment 210 can be any volume between about 0 and about 10 ml, e.g., about 0 ml, 0.1 ml, 0.2 ml, 0.3 ml, 0.4 ml, 0.5 ml, 0.6 ml, 0.7 ml, 0.8 ml, 0.9 ml, 1.0 ml, 1.1 ml, 1.2 ml, 1.3 ml, 1.4 ml, 1.5 ml, 1.6 ml, 1.7 ml, 1.8 ml, 1.9 ml, 2.0 ml, 2.1 ml, 2.2 ml, 2.3 ml, 2.4 ml, 2.5 ml, 2.6 ml, 2.7 ml, 2.8 ml, 2.9 ml, 3.0 ml, 3.1 ml, 3.2 ml, 3.3 ml, 3.4 ml, 3.5 ml, 3.6 ml, 3.7 ml, 3.8 ml, 3.9 ml, 4.0 ml, 4.1 ml, 4.2 ml, 4.3 ml, 4.4 ml, 4.5 ml, 4.6 ml, 4.7 ml, 4.8 ml, 4.9 ml, 5.0 ml, 5.1 ml, 5.2 ml, 5.3 ml, 5.4 ml, 5.5 ml, 5.6 ml, 5.7 ml, 5.8 ml, 5.9 ml, 6.0 ml, 6.1 ml, 6.2 ml, 6.3 ml, 6.4 ml, 6.5 ml, 6.6 ml, 6.7 ml, 6.8 ml, 6.9 ml, 7.0 ml, 7.1 ml, 7.2 ml, 7.3 ml, 7.4 ml, 7.5 ml, 7.6 ml, 7.7 ml, 7.8 ml, 7.9 ml, 8.0 ml, 8.1 ml, 8.2 ml, 8.3 ml, 8.4 ml, 8.5 ml, 8.6 ml, 8.7 ml, 8.8 ml, 8.9 ml, 9.0 ml, 9.1 ml, 9.2 ml, 9.3 ml, 9.4 ml, 9.5 ml, 9.6 ml, 9.7 ml, 9.8 ml, 9.9 ml, or about 10.0 ml, or any amount therebetween.

In some embodiments, the predefined volume of the metering compartment 210 can be any volume between about 0 and about 1.0 ml, between about 0 and about 0.9 ml, between about 0 and about 0.8 ml, between about 0 and about 0.7 ml, between about 0 and about 0.6 ml, between about 0 and about 0.5 ml, between about 0 and about 0.4 ml, between about 0 and about 0.3 ml. between about 0 and about 0.2 ml, between about 0 and about 0.1 ml, between about 0.1 and about 1.0 ml, between about 0.1 and about 0.9 ml, between about 0.1 and about 0.8 ml, between about 0.1 and about 0.7 ml, between about 0.1 and about 0.6 ml, between about 0.1 and about 0.5 ml, between about 0.1 and about 0.4 ml, between about 0.1 and about 0.3 ml, between about 0.1 and about 0.2 ml, between about 0.2 and about 1.0 ml, between about 0.2 and about 0.9 ml, between about 0.2 and about 0.8 ml, between about 0.2 and about 0.7 ml, between about 0.2 and about 0.6 ml, between about 0.2 and about 0.5 ml, between about 0.2 and about 0.4 ml, between about 0.2 and about 0.3 ml, between about 0.3 and about 1.0 ml, between about 0.3 and about 0.9 ml, between about 0.3 and about 0.8 ml, between about 0.3 and about 0.7 ml, between about 0.3 and about 0.6 ml, between about 0.3 and about 0.5 ml, between about 0.3 and about 0.4 ml, between about 0.4 and about 1.0 ml, between about 0.4 and about 0.9 ml, between about 0.4 and about 0.8 ml, between about 0.4 and about 0.7 ml, between about 0.4 and about 0.6 ml, between about 0.4 and about 0.5 ml, between about 0.5 and about 1.0 ml, between about 0.5 and about 0.9 ml, between about 0.5 and about 0.8 ml, between about 0.5 and about 0.7 ml, between about 0.5 and about 0.6 ml, between about 0.6 and about 1.0 ml, between about 0.6 and about 0.9 ml, between about 0.6 and about 0.8 ml, between about 0.6 and about 0.7 ml, between about 0.7 and about 1.0 ml, between about 0.7 and about 0.9 ml, between about 0.7 and about 0.8 ml, between about 0.8 and about 1.0 ml, between about 0.8 and about 0.9 ml, or between about 0.9 and about 1.0 ml.

In some embodiments, the predefined volume of the metering compartment 210 can be any volume between about 0 and about 1.0 ml, e.g., about 0.01 ml, 0.02 ml, 0.03 ml, 0.04 ml, 0.05 ml, 0.06 ml, 0.07 ml, 0.08 ml, 0.09 ml, 0.10 ml, 0.11 ml, 0.12 ml, 0.13 ml, 0.14 ml, 0.15 ml, 0.16 ml, 0.17 ml, 0.18 ml, 0.19 ml, 0.20 ml, 0.21 ml, 0.22 ml, 0.23 ml, 0.24 ml, 0.25 ml, 0.26 ml, 0.27 ml, 0.28 ml, 0.29 ml, 0.30 ml, 0.31 ml, 0.32 ml, 0.33 ml, 0.34 ml, 0.35 ml, 0.36 ml, 0.37 ml, 0.38 ml, 0.39 ml, 0.40 ml, 0.41 ml, 0.42 ml, 0.43 ml, 0.44 ml, 0.45 ml, 0.46 ml, 0.47 ml, 0.48 ml, 0.49 ml, 0.50 ml, 0.51 ml, 0.52 ml, 0.53 ml, 0.54 ml, 0.55 ml, 0.56 ml, 0.57 ml, 0.58 ml, 0.59 ml, 0.60 ml, 0.61 ml, 0.62 ml, 0.63 ml, 0.64 ml, 0.65 ml, 0.66 ml, 0.67 ml, 0.68 ml, 0.69 ml, 0.70 ml, 0.71 ml, 0.72 ml, 0.73 ml, 0.74 ml, 0.75 ml, 0.76 ml, 0.77 ml, 0.78 ml, 0.79 ml, 0.80 ml, 0.81 ml, 0.82 ml, 0.83 ml, 0.84 ml, 0.85 ml, 0.86 ml, 0.87 ml, 0.88 ml, 0.89 ml, 0.90 ml, 0.91 ml, 0.92 ml, 0.93 ml, 0.94 ml, 0.95 ml, 0.96 ml, 0.97 ml, 0.98 ml, 0.99 ml, or about 1.0 ml, or any amount therebetween.

In some embodiments, the predefined volume of the metering compartment 210 can be any volume between about 0 and about 0.10 ml, between about 0 and about 0.09 ml, between about 0 and about 0.08 ml, between about 0 and about 0.07 ml, between about 0 and about 0.06 ml, between about 0 and about 0.05 ml, between about 0 and about 0.04 ml, between about 0 and about 0.03 ml, between about 0 and about 0.02 ml, between about 0 and about 0.01 ml, between about 0.01 and 0.10 ml, between about 0.01 and about 0.09 ml, between about 0.01 and about 0.08 ml, between about 0.01 and about 0.07 ml, between about 0.01 and about 0.06 ml, between about 0.01 and about 0.05 ml, between about 0.01 and about 0.04 ml, between about 0.01 and about 0.03 ml, between about 0.01 and about 0.02 ml, between about 0.02 and about 0.10 ml, between about 0.02 and about 0.09 ml, between about 0.02 and about 0.08 ml, between about 0.02 and about 0.07 ml, between about 0.02 and about 0.06 ml, between about 0.02 and about 0.05 ml, between about 0.02 and about 0.04 ml, between about 0.02 and about 0.03 ml, between about 0.03 and about 0.10 ml, between about 0.03 and about 0.09 ml, between about 0.03 and about 0.08 ml, between about 0.03 and about 0.07 ml, between about 0.03 and about 0.06 ml, between about 0.03 and about 0.05 ml, between about 0.03 and about 0.04 ml, between about 0.04 and about 0.10 ml, between about 0.04 and about 0.09 ml, between about 0.04 and about 0.08 ml, between about 0.04 and about 0.07 ml, between about 0.04 and about 0.06 ml, between about 0.04 and about 0.05 ml, between about 0.05 and about 0.10 ml, between about 0.05 and about 0.09 ml, between about 0.05 and about 0.08 ml, between about 0.05 and about 0.07 ml, between about 0.05 and about 0.06 ml, between about 0.06 and about 0.10 ml, between about 0.06 and about 0.09 ml, between about 0.06 and about 0.08 ml, between about 0.06 and about 0.07 ml, between about 0.07 and about 0.10 ml, between about 0.07 and about 0.09 ml, between about 0.07 and about 0.08 ml, between about 0.08 and about 0.10 ml, between about 0.08 and about 0.09 ml, or between about 0.09 and about 0.1 ml.

In some embodiments, the predefined volume of the metering compartment 210 can be any volume between about 0 and about 0.1 ml, e.g., about 0.001 ml, 0.002 ml, 0.003 ml, 0.004 ml, 0.005 ml, 0.006 ml, 0.007 ml, 0.008 ml, 0.009 ml, 0.010 ml, 0.011 ml, 0.012 ml, 0.013 ml, 0.014 ml, 0.015 ml, 0.016 ml, 0.017 ml, 0.018 ml, 0.019 ml, 0.020 ml, 0.021 ml, 0.022 ml, 0.023 ml, 0.024 ml, 0.025 ml, 0.026 ml, 0.027 ml, 0.028 ml, 0.029 ml, 0.030 ml, 0.031 ml, 0.032 ml, 0.033 ml, 0.034 ml, 0.035 ml, 0.036 ml, 0.037 ml, 0.038 ml, 0.039 ml, 0.040 ml, 0.041 ml, 0.042 ml, 0.043 ml, 0.044 ml, 0.045 ml, 0.046 ml, 0.047 ml, 0.048 ml, 0.049 ml, 0.050 ml, 0.051 ml, 0.052 ml, 0.053 ml, 0.054 ml, 0.055 ml, 0.056 ml, 0.057 ml, 0.058 ml, 0.059 ml, 0.060 ml, 0.061 ml, 0.062 ml, 0.063 ml, 0.064 ml, 0.065 ml, 0.066 ml, 0.067 ml, 0.068 ml, 0.069 ml, 0.070 ml, 0.071 ml, 0.072 ml, 0.073 ml, 0.074 ml, 0.075 ml, 0.076 ml, 0.077 ml, 0.078 ml, 0.079 ml, 0.008 ml, 0.081 ml, 0.082 ml, 0.083 ml, 0.084 ml, 0.085 ml, 0.086 ml, 0.087 ml, 0.088 ml, 0.089 ml, 0.090 ml, 0.091 ml, 0.092 ml, 0.093 ml, 0.094 ml, 0.095 ml, 0.096 ml, 0.097 ml, 0.098 ml, 0.099 ml, or about 0.100 ml, or any volume therebetween.

In some embodiments, the predefined volume of the biological sample can be driven or otherwise flow (e.g., under capillary effect) from the metering compartment 210 through a reagent storage zone 214 to the reaction chamber 208. The reagent storage zone 214 can include one or more reagents, for example, reagents useful in processing the biological sample. In some embodiments, the reagent may be in dried form and rehydrated as a liquid biological sample flows through the reagent storage zone 214. Where analysis of nucleic acids from the biological sample is desired, the reagent storage zone can include reagents for nucleic acid extraction (e.g., extraction of RNA, DNA, gDNA, mtDNA, bacterial DNA, mRNA, miRNA, cfRNA, cfDNA, ribosomal RNA, etc) from the biological sample. The reagent storage zone 214 can include any suitable amount or concentration of reagent needed for nucleic acid extraction. In some embodiments, the amount or concentration of reagent depends on the volume of biological sample to be processed. The volume of the biological sample to be processed may be determined by the metering compartment 210.

In some embodiments, the mixture of biological sample and one or more reagents may flow to the reaction chamber 208 where it may mix with one or more additional reagents. In some embodiments, the additional reagent may be stored in the blister 106. The mixture of biological sample and reagents may undergo one or more processing steps in the plurality of reaction compartments (e.g., 208A, 208B). One or more blisters in the reagent storage zone 214 can include wet reagents needed to process the biological sample, for example extract nucleic acids. Reagents include, but are not limited to, lysis, wash, and reaction buffers with the appropriate concentrations of salts, surfactants, enzymes, and/or solvents such as ethanol and isopropanol. In some embodiments, the one or more wet reagents may flow from the blisters to a reaction compartment 208 through a channel of the fluidic device. In some embodiments, the wet reagent may flow out of the blister 106 when the blister 106 is compressed by a mechanical member located inside the instrument and the blister is ruptured. The different reagents stored in the wet blisters can be flown into the reaction chamber 208 as needed to complete the steps of the biological sample processing. In some embodiments, a predefined volume of a wet reagent may flow or be driven from the blister 106 through the metering compartment 210 to the reaction chamber 208.

In some embodiments, the reaction chamber 208 can be coupled to a temperature control subsystem 340 (described in more detail below). The temperature control subsystem 303 can be configured to maintain the reaction chamber 208 at a reaction chamber temperature for performance of an assay. In some embodiments, the temperature control subsystem 340 can be incorporated into the instrument 300, as shown in FIGS. 1 and 3. In some embodiments, the reaction chamber temperature may be any temperature between about 37° C. and about 95° C., e.g., about 37° C., 38° C., 39° C., 40° C., 41° C., 42° C., 43° C., 44° C., 45° C., 4° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., or about 95° C., or any temperature therebetween. Adjusting the temperature of the reaction chamber may be useful in accomplishing one or more steps of the biological sample processing. For example, heating the biological sample to a temperature between about 55° C. and about 75° C. may be useful for cell lysis during nucleic acid extraction. Heating the biological sample to a temperature between 37° C. and 55° C. may be useful for optimal activity of enzymes used to process the biological sample. For example, the reaction chamber may be increased to about 55° C. for optimal activity of proteases during nucleic acid extraction. The temperature control subsystem 303 can control temperature for isothermal amplification operations, polymerase chain reaction, and/or other amplification processes. The temperature control subsystem 303 can control temperature for isothermal cleavage of thermally-activated molecule regions of functionalized molecules, functionalized particles, functionalized substrates, etc.

The temperature control subsystem 340 can include a resistance heater, a Peltier heater, a radiative heating element, a conductive heating element, a convective heating element, or other suitable heating element that is thermally coupled to the fluidic device 100 (e.g., at reaction chamber 108 or other portion) in association with performance of a reaction requiring controlled heat modulation.

In some embodiments, methods executed by the system 10 can involve extracting nucleic acids of the biological sample using magnetic beads, such as magnetic beads functionalized with oligonucleotides capable of extracting nucleic acids from the biological sample. In embodiments, the nucleic acid of interest from the biological sample can be mRNA and the magnetic beads can be coupled to an oligo-deoxythymidine (oligo-dT) which is capable of hybridizing to mRNA through pairing with the poly-adenosine monophosphates (polyA) tail of the mRNA. Alternatively, the magnetic beads can be coupled to a target-specific capture sequence configured to bind to or otherwise preferentially interact with a loci of interest in the target(s) of the biological sample. The reagent storage zone 106 can store the magnetic beads as well as additional reagents needed to extract the nucleic acids. Additional reagents include, but are not limited to, wash buffers to remove undesired nucleic acids from the magnetic beads and elution buffers to collect the desired nucleic acids from the magnetic beads for further analysis.

In embodiments, magnetic beads can include polymer beads, pre-coupled with an molecule for binding to a portion of a nucleic acid of interest from the biological sample, and including a magnetic component (e.g., a magnetic, parmagnetic, or superparamagnetic component). Additionally, the magnetic beads may be treated to be positively charged. However, the magnetic beads may alternatively be any appropriate magnetic beads configured to facilitate biomagnetic separation.

Alternatively, buoyant beads, non-magnetic beads, or other beads can be implemented for attracting sample components (or waste components), in order to facilitate detection of expression levels of biomarkers of interest from the biological sample.

In some embodiments, the reaction chamber 208 may be operatively coupled to a magnetic field. In some embodiments, the magnetic field may be generated by the instrument. In some embodiments, the magnetic bead in the reaction chamber may be operatively coupled to the magnetic field. The magnetic field may be configured to immobilize the magnetic bead in the reaction chamber 208 during the nucleic acid extraction process. The magnetic field can be governed by one or more magnets of the system 10 (e.g., included in instrument 300), and can be reversible, electromagnetically applied, or otherwise applied.

In some embodiments, the reaction compartments of the reaction chamber may be configured to be in fluid communication. In some embodiments, the reaction compartments may be configured such that the movement of liquid between the reaction compartments is well controlled (e.g., minimal leaking or mixing between compartments).

Depending on the further analysis to be completed with the extracted nucleic acid, a minimum amount of nucleic acid may be desired. In some embodiments, the concentration of nucleic acid extracted from the biological sample is between about 0.1 ng/L and about 10.0 ng/L, e.g., about 0.1 ng/L, 0.2 ng/L, 0.3 ng/L, 0.4 ng/L, 0.5 ng/L, 0.6 ng/L, 0.07 ng/L, 0.8 ng/L, 0.9 ng/L, 1.0 ng/L, 1.1 ng/L, 1.2 ng/L, 1.3 ng/L, 1.4 ng/L, 1.5 ng/L, 1.6 ng/L, 1.7 ng/L, 1.8 ng/L, 1.9 ng/L, 2.0 ng/L, 2.1 ng/L, 2.2 ng/L, 2.3 ng/L, 2.4 ng/L, 2.5 ng/L, 2.6 ng/L, 2.7 ng/L, 2.8 ng/L, 2.9 ng/L, 3.0 ng/L, 3.1 ng/L, 3.2 ng/L, 3.3 ng/L, 3.4 ng/L, 3.5 ng/L, 3.6 ng/L, 3.7 ng/L, 3.8 ng/L, 3.9 ng/L, 4.0 ng/L, 4.1 ng/L, 4.2 ng/L, 4.3 ng/L, 4.4 ng/L, 4.5 ng/L, 4.6 ng/L, 4.7 ng/L, 4.8 ng/L, 4.9 ng/L, 5.0 ng/L, 5.1 ng/L, 5.2 ng/L, 5.3 ng/L, 5.4 ng/L, 5.5 ng/L, 5.6 ng/L, 5.7 ng/L, 5.8 ng/L, 5.9 ng/L, 6.0 ng/L, 6.1 ng/L, 6.2 ng/L, 6.3 ng/L, 6.4 ng/L, 6.5 ng/L, 6.6 ng/L, 6.7 ng/L, 6.8 ng/L, 6.9 ng/L, 7.0 ng/L, 7.1 ng/L, 7.2 ng/L, 7.3 ng/L, 7.4 ng/L, 7.5 ng/L, 7.6 ng/L, 7.7 ng/L, 7.8 ng/L, 7.9 ng/L, 8.0 ng/L, 8.1 ng/L, 8.2 ng/L, 8.3 ng/L, 8.4 ng/L, 8.5 ng/L, 8.6 ng/L, 8.7 ng/L, 8.8 ng/L, 8.9 ng/L, 9.0 ng/L, 9.1 ng/L, 9.2 ng/L, 9.3 ng/L, 9.4 ng/L, 9.5 ng/L, 9.6 ng/L, 9.7 ng/L, 9.8 ng/L, 9.9 ng/L, or about 10.0 ng/L, or any amount therebetween. In some embodiments, the concentration of nucleic acid extracted from the biological sample is between about 10 ng/L and about 100 ng/L, e.g., about 10 ng/L, 11 ng/L, 12 ng/L, 13 ng/L, 14 ng/L, 15 ng/L, 16 ng/L, 17 ng/L, 18 ng/L, 19 ng/L, 20 ng/L, 21 ng/L, 22 ng/L, 23 ng/L, 24 ng/L, 25 ng/L, 26 ng/L, 27 ng/L, 28 ng/L, 29 ng/L, 30 ng/L, 31 ng/L, 32 ng/L, 33 ng/L, 34 ng/L, 35 ng/L, 36 ng/L, 37 ng/L, 38 ng/L, 39 ng/L, 40 ng/L, 41 ng/L, 42 ng/L, 43 ng/L, 44 ng/L, 45 ng/L, 46 ng/L, 47 ng/L, 48 ng/L, 49 ng/L, 50 ng/L, 51 ng/L, 52 ng/L, 53 ng/L, 54 ng/L, 55 ng/L, 56 ng/L, 57 ng/L, 58 ng/L, 59 ng/L, 60 ng/L, 61 ng/L, 62 ng/L, 63 ng/L, 64 ng/L, 65 ng/L, 66 ng/L, 67 ng/L, 68 ng/L, 69 ng/L, 70 ng/L, 71 ng/L, 72 ng/L, 73 ng/L, 74 ng/L, 75 ng/L, 76 ng/L, 77 ng/L, 78 ng/L, 79 ng/L, 80 ng/L, 81 ng/L, 82 ng/L, 83 ng/L, 84 ng/L, 85 ng/L, 86 ng/L, 87 ng/L, 88 ng/L, 89 ng/L, 90 ng/L, 91 ng/L, 92 ng/L, 93 ng/L, 94 ng/L, 95 ng/L, 96 ng/L, 97 ng/L, 98 ng/L, 99 ng/L, or about 100 ng/L, or any amount therebetween. In some embodiments, the amount of nucleic acid extracted from the biological sample is between about 100 ng/L and about 1 microgram/L (μg/L), e.g., about 100 ng/L, 110 ng/L, 120 ng/L, 130 ng/L, 140 ng/L, 150 ng/L, 160 ng/L, 170 ng/L, 180 ng/L, 190 ng/L, 200 ng/L, 210 ng/L, 220 ng/L, 230 ng/L, 240 ng/L, 250 ng/L, 260 ng/L, 270 ng/L, 280 ng/L, 290 ng/L, 300 ng/L, 310 ng/L, 320 ng/L, 330 ng/L, 340 ng/L, 350 ng/L, 360 ng/L, 370 ng/L, 380 ng/L, 390 ng/L, 400 ng/L, 410 ng/L, 420 ng/L, 430 ng/L, 440 ng/L, 450 ng/L, 460 ng/L, 470 ng/L, 480 ng/L, 490 ng/L, 500 ng/L, 510 ng/L, 520 ng/L, 530 ng/L, 540 ng/L, 550 ng/L, 560 ng/L, 570 ng/L, 580 ng/L, 590 ng/L, 600 ng/L, 610 ng/L, 620 ng/L, 630 ng/L, 640 ng/L, 650 ng/L, 660 ng/L, 670 ng/L, 680 ng/L, 690 ng/L, 700 ng/L, 710 ng/L, 720 ng/L, 730 ng/L, 740 ng/L, 750 ng/L, 760 ng/L, 770 ng/L, 780 ng/L, 790 ng/L, 800 ng/L, 810 ng/L, 820 ng/L, 830 ng/L, 840 ng/L, 850 ng/L, 860 ng/L, 870 ng/L, 880 ng/L, 890 ng/L, 900 ng/L, 910 ng/L, 920 ng/L, 930 ng/L, 940 ng/L, 950 ng/L, 960 ng/L, 970 ng/L, 980 ng/L, 990 ng/L, or about 1000 ng/L, or any amount therebetween. In some embodiments, the amount of nucleic acid disclosed above may be applicable to any type of nucleic acid including, but not limited to, a DNA, a cDNA, a RNA, a mRNA, a miRNA, a cfDNA, a cfRNA, etc.

In some embodiments, the amount of nucleic acid extracted from the biological sample is at least 0.01 ng/L, at least 0.02 ng/L, at least 0.03 ng/L, 0.04 ng/L, at least 0.05 ng/L, at least 0.06 ng/L, at least 0.07 ng/L, 0.08 ng/L, at least 0.09 ng/L, at least 0.10 ng/L, at least 0.20 ng/L, at least 0.30 ng/L, at least 0.40 ng/L, at least 0.50 ng/L, at least 0.60 ng/L, at least 0.70 ng/L, at least 0.80 ng/L, at least 0.90 ng/L, at least 1.0 ng/L, at least 2.0 ng/L, at least 3.0 ng/L, at least 4.0 ng/L, at least 5.0 ng/L, at least 6.0 ng/L, at least 7.0 ng/L, at least 8.0 ng/L, at least 9.0 ng/L, at least 10 ng/L, at least 15 ng/L, at least 20 ng/L, at least 25 ng/L, at least 30 ng/L, at least 35 ng/L, at least 40 ng/L, at least 45 ng/L, at least 50 ng/L, at least 55 ng/L, at least 60 ng/L ng/L, at least 65 ng/L, at least 70 ng/L, at least 75 ng/L, at least 80 ng/L, at least 85 ng/L, at least 90 ng/L, at least 95 ng/L, at least 100 ng/L, at least 200 ng/L, at least 300 ng/L, at least 400 ng/L, at least 500 ng/L, at least 600 ng/L, at least 700 ng/L, at least 800 ng/L, at least 900 ng/L, or at least 1000 ng/L.

In some embodiments, the nucleic acid extracted from the biological sample is subject to a detection reaction to measure biomarker expression levels, according to the set of processes described in relation to method 500. For example, the detection reaction may comprise amplification of the nucleic acids within the fluidic device. One or more reagents for amplification or quantitation of biomarker expression can be stored in a reagent storage zone. In some embodiments, the nucleic acid solution rehydrates one or more dried reagents (e.g., amplification or detection reagents) stored in a reagent storage zone 214 as it flows to a detection chamber 216 in the fluidic device.

In some embodiments, the detection reaction, as one of a set of processes of method 500 (described below) can include first reverse transcribing mRNA to cDNA and then amplifying the cDNA. In some embodiments, the nucleic acid is amplified in a quantitative manner. Any suitable amplification method can be employed. In some embodiments, the amplification method may comprise isothermal amplification. In some embodiments, the isothermal amplification may comprise a loop-mediated isothermal amplification (LAMP). LAMP offers selectivity and employs a polymerase and a set of specifically designed primers that recognize distinct sequences in a target nucleic acid. Unlike PCR, the target nucleic acid is amplified at a constant temperature (e.g., 60-65° C.) using multiple inner and outer primers and a polymerase having strand displacement activity. In some instances, an inner primer pair containing a nucleic acid sequence complementary to a portion of the sense and antisense strands of the target nucleic acid initiate LAMP. Following strand displacement synthesis by the inner primers, strand displacement synthesis primed by an outer primer pair can cause release of a single-stranded amplicon. The single-stranded amplicon may serve as a template for further synthesis primed by a second inner and second outer primer that hybridize to the other end of the target nucleic acid and produce a stem-loop nucleic acid structure. In subsequent LAMP cycling, one inner primer hybridizes to the loop on the product and initiates displacement and target nucleic acid synthesis, yielding the original stem-loop product and a new stem-loop product with a stem twice as long. Additionally, the 3′ terminus of an amplicon loop structure serves as initiation site for self-templating strand synthesis, yielding a hairpin-like amplicon that forms an additional loop structure to prime subsequent rounds of self-templated amplification. The amplification continues with accumulation of many copies of the target nucleic acid. The final products of the LAMP process are stem-loop nucleic acids with concatenated repeats of the target nucleic acid in cauliflower-like structures with multiple loops formed by annealing between alternately inverted repeats of a target nucleic acid sequence in the same strand.

In some embodiments, the isothermal amplification assay comprises a digital reverse-transcription loop-mediated isothermal amplification (dRT-LAMP) reaction for quantifying the target nucleic acid. Typically, LAMP assays produce a detectable signal (e.g., fluorescence) during the amplification reaction. In some embodiments, fluorescence can be detected and quantified.

In some embodiments, the amplification reaction comprises polymerase chain reaction (PCR) amplification, recombinase polymerase amplification (RPA), ligase chain reaction, branched DNA amplification, nucleic acid sequence-based amplification (NASBA), strand displacement assay (SDA), transcription-mediated amplification, rolling circle amplification, helicase-dependent amplification (HDA), single primer isothermal amplification (SPIA), nicking and extension amplification reaction (NEAR), or transcription mediated assay (TMA).

In some embodiments, the nucleic acid extracted from the biological sample is subjected to a clustered regularly interspaced short palindromic repeats-Cas (CRISPR-Cas) reaction to determine the expression levels of nucleic acid biomarkers in the biological sample.

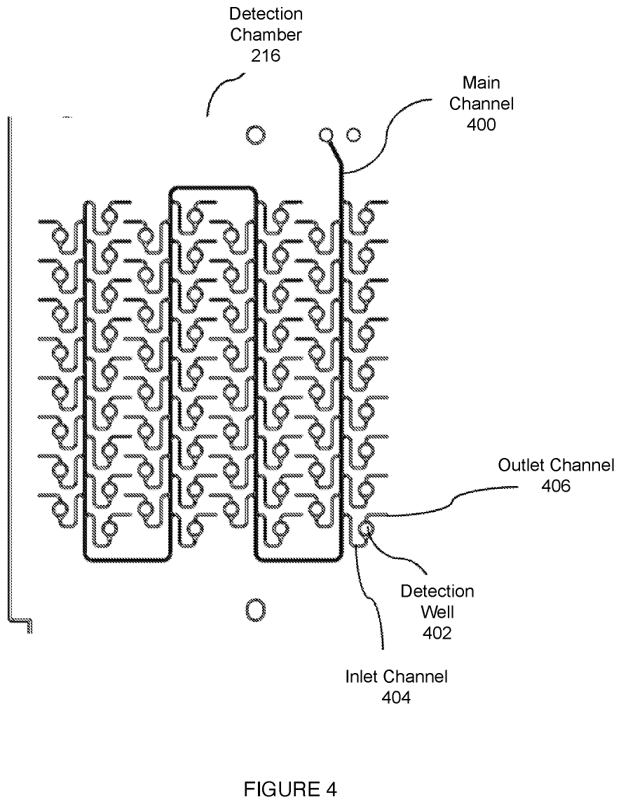

In some embodiments, the fluidic device comprises a detection chamber 216 downstream of and fluidically coupled to reaction chamber 208. The detection chamber 216 can include comprise a plurality of detection wells, as shown in FIG. 4. In some embodiments, the plurality of detection wells can be connected by a main channel 400. The main channel 400 can be configured to transmit fluid derived from the biological sample, reagents, and/or other materials to the plurality of detection wells sequentially. The main channel 400 can be in fluid communication with other channels of the fluidic device 100 and can move extracted nucleic acids, e.g., from the reaction chamber 208 to the detection chamber 216. In some aspects, the extracted nucleic acid passes through a reagent storage zone 214 to rehydrate dried reagents for the detection reaction. In cases where the detection reaction is nucleic acid amplification, the dried reagents can include polymerase, a dye, and one or more buffer ingredients. An individual detection well 402 can be connected to the main channel by an inlet channel 404. In some embodiments, all detection wells are connected to the main channel 400 by a respective inlet channel. In some cases, inlet channels of the detection chamber 216 are all approximately the same length. In preferred embodiments, the length of the inlet channels are sufficient to minimize back-diffusion of primers from the detection wells into the main channel as the detection wells are being filled. An individual detection well can be connected to an outlet channel 406. Each detection well may be connected to an outlet channel. The outlet channel 406 may comprise a vent at the terminus. The vent can be permeable to gas but not liquid. The presence of a gas permeable vent may allow the detection well to efficiently fill with extracted nucleic acid. In some embodiments, each outlet channel is vented at the terminus. In some embodiments, all detection wells each have an outlet channel which is vented at the terminus. In some embodiments, a plurality of vents at outlet channels are connected to a master vent which can open and close all outlet vents simultaneously. In some embodiments, the detection chamber is covered by a structure that prevents evaporation during nucleic acid amplification from the vents.

In the case of nucleic acid amplification for biomarker quantification, each of the detection wells can contain a primer or primer set suitable for amplifying mRNA with a particular gene sequence from the biological sample. The one or more detection wells can be in fluid communication through a channel with other components of the fluidic device 100. For example, the detection chamber 216 can be in fluid communication with the reagent storage zone 214 such that a solution containing extracted nucleic acid flows through the reagent storage zone to rehydrate one or more reagents before entering into the detection well. For a given biomarker, a plurality of primers (e.g., a primer set) may be used for detection by amplification, such that the fluidic device 100 can be configured to perform amplification with a high degree of multiplexing for different biomarkers of the sample, in parallel. Where amplification of mRNA is desired without amplification of genomic DNA of a biomarker, the primer set can be designed in such a way that the mRNA is selectively amplified. For example, the primer or primer set may be derived only from one or more exon regions of a gene but not from an intron region of a gene. An exon is part of a gene that will form the final product of a mature mRNA while an intron is part of a gene that is removed during the processing of an mRNA and hence does not form the final product of a mature mRNA. Primers that target exon-exon junctions may specifically amplify mRNA (and corresponding cDNA) without amplifying genomic DNA.

The fluidic device 100 can thus be configured to perform multiplexed amplification, in parallel, of: 2 targets, 3 targets, 4 targets, 5 targets, 6 targets, 7 targets, 8 targets, 9 targets, 10 targets, 11 targets, 12 targets, 13 targets, 14 targets, 15 targets, 16 targets, 17 targets, 18 targets, 19 targets, 20 targets, 30 targets, 40 targets, 50 targets, 60 targets, 70 targets, 80 targets, 90 targets, 100 targets, or more (e.g., using a LAMP-based approach, using another approach for amplification of nucleic acid targets, etc.).

In some embodiments, a detection well 216 can contain a first primer or primer set for amplification of a first gene and a second well may contain a second primer or primer set for amplification of a second gene. In some embodiments, the first gene and the second gene may be different. In some embodiments, the first gene and the second gene may be the same and the two detection wells are used as duplicates. In some embodiments, the first and second primers or primer sets may be configured so that the amplification efficiency of the first gene and second gene are comparable. In some cases, a detection well may not comprise primers for a biomarker gene but contains primers for a housekeeping gene which can be used for quality control.

In some embodiments, the detection chamber 216 can include at least two detection wells each of which contains a primer or primer set for a different gene. In some embodiments, the detection chamber may comprise at least two wells each of which contains a primer sequence or primer sequences for the same gene. In some embodiments, the fluidic device comprises a plurality of detection wells, such as at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more than 50 detection wells. The presence of many detection wells in the detection chamber allows for a high-throughput analysis of many genes. In some embodiments, the gene may comprise a biomarker for diagnosis of a disease, disorder, or condition. In some embodiments, the gene may comprise a housekeeping gene. In some embodiments, the gene may comprise an internal control nucleic acid sequence. In some embodiments, the gene may have more than one function. As such, in examples, the system provides structures for split-reaction multi-well LAMP reactions in an unprecedented manner, with respect to performance, and at the point-of-care.

In some embodiments, the concentration of the primer or primer set in a detection well may be between 0 μM and 10 μM, e.g., about 0.001 μM, 0.002 μM, 0.003 μM, 0.004 μM, 0.005 μM, 0.006 μM, 0.007 μM, 0.008 μM, 0.009 μM, 0.010 μM, 0.011 μM, 0.012 μM, 0.013 μM, 0.014 μM, 0.015 μM, 0.016 μM, 0.017 μM, 0.018 μM, 0.019 μM, 0.020 μM, 0.021 μM, 0.022 μM, 0.023 μM, 0.024 μM, 0.025 μM, 0.026 μM, 0.027 μM, 0.028 μM, 0.029 μM, 0.030 μM, 0.031 μM, 0.032 μM, 0.033 μM, 0.034 μM, 0.035 μM, 0.036 μM, 0.037 μM, 0.038 μM, 0.039 μM, 0.040 μM, 0.041 μM, 0.042 μM, 0.043 μM, 0.044 μM, 0.045 μM, 0.046 μM, 0.047 μM, 0.048 μM, 0.049 μM, 0.050 μM, 0.051 μM, 0.052 μM, 0.053 μM, 0.054 μM, 0.055 μM, 0.056 μM, 0.057 μM, 0.058 μM, 0.059 μM, 0.060 μM, 0.061 μM, 0.062 μM, 0.063 μM, 0.064 μM, 0.065 μM, 0.066 μM, 0.067 μM, 0.068 μM, 0.069 μM, 0.070 μM, 0.071 μM, 0.072 μM, 0.073 μM, 0.074 μM, 0.075 μM, 0.076 μM, 0.077 μM, 0.078 μM, 0.079 μM, 0.080 μM, 0.081 μM, 0.082 μM, 0.083 μM, 0.084 μM, 0.085 μM, 0.086 μM, 0.087 μM, 0.088 μM, 0.089 μM, 0.090 μM, 0.091 μM, 0.092 μM, 0.093 μM, 0.094 μM, 0.095 μM, 0.096 μM, 0.097 μM, 0.098 μM, 0.099 μM, 0.100 μM, 0.110 μM, 0.120 μM, 0.130 μM, 0.140 μM, 0.150 μM, 0.160 μM, 0.170 μM, 0.180 μM, 0.190 μM, 0.200 μM, 0.210 μM, 0.220 μM, 0.230 μM, 0.240 μM, 0.250 μM, 0.260 μM, 0.270 μM, 0.280 μM, 0.290 μM, 0.300 μM, 0.310 μM, 0.320 μM, 0.330 μM, 0.340 μM, 0.350 μM, 0.360 μM, 0.370 μM, 0.380 μM, 0.390 μM, 0.400 μM, 0.410 μM, 0.420 μM, 0.430 μM, 0.440 μM, 0.450 μM, 0.460 μM, 0.470 μM, 0.480 μM, 0.490 μM, 0.500 μM, 0.510 μM, 0.520 μM, 0.530 μM, 0.540 μM, 0.550 μM, 0.560 μM, 0.570 μM, 0.580 μM, 0.590 μM, 0.600 μM, 0.610 μM, 0.620 μM, 0.630 μM, 0.640 μM, 0.650 μM, 0.660 μM, 0.670 μM, 0.680 μM, 0.690 μM, 0.700 μM, 0.710 μM, 0.720 μM, 0.730 μM, 0.740 μM, 0.750 μM, 0.760 μM, 0.770 μM, 0.780 μM, 0.790 μM, 0.800 μM, 0.810 μM, 0.820 μM, 0.830 μM, 0.840 μM, 0.850 μM, 0.860 μM, 0.870 μM, 0.880 μM, 0.890 μM, 0.900 μM, 0.910 μM, 0.920 μM, 0.930 μM, 0.940 μM, 0.950 μM, 0.960 μM, 0.970 μM, 0.980 μM, 0.990 μM, 1.00 μM, 1.10 μM, 1.20 μM, 1.30 μM, 1.40 μM, 1.50 μM, 1.60 μM, 1.70 μM, 1.80 μM, 1.90 μM, 2.00 μM, 2.10 μM, 2.20 μM, 2.30 μM, 2.40 μM, 2.50 μM, 2.60 μM, 2.70 μM, 2.80 μM, 2.90 μM, 3.00 μM, 3.10 μM, 3.20 μM, 3.30 μM, 3.40 μM, 3.50 μM, 3.60 μM, 3.70 μM, 3.80 μM, 3.90 μM, 4.00 μM, 4.10 μM, 4.20 μM, 4.30 μM, 4.40 μM, 4.50 μM, 4.60 μM, 4.70 μM, 4.80 μM, 4.90 μM, 5.00 μM, 5.10 μM, 5.20 μM, 5.30 μM, 5.40 μM, 5.50 μM, 5.60 μM, 5.70 μM, 5.80 μM, 5.90 μM, 6.00 μM, 6.10 μM, 6.20 μM, 6.30 μM, 6.40 μM, 6.50 μM, 6.60 μM, 6.70 μM, 6.80 μM, 6.90 μM, 7.00 μM, 7.10 μM, 7.20 μM, 7.30 μM, 7.40 μM, 7.50 μM, 7.60 μM, 7.70 μM, 7.80 μM, 7.90 μM, 8.00 μM, 8.10 μM, 8.20 μM, 8.30 μM, 8.40 μM, 8.50 μM, 8.60 μM, 8.70 μM, 8.80 μM, 8.90 μM, 9.00 μM, 9.10 μM, 9.20 μM, 9.30 μM, 9.40 μM, 9.50 μM, 9.60 μM, 9.70 μM, 9.80 μM, 9.90 μM, or about 10.00 μM or any concentration therebetween.

Individual detection wells of a fluidic device 100 can have any volume suitable for carrying out detection of biomarkers in a biological sample. In some embodiments, the volume of individual detection wells 216 is suitable for conducting amplification reactions. In some embodiments, the volumes of all the detection wells within a detection chamber are the same. In some embodiments, some of the detection wells have a first volume and some of the detection wells have a second volume. The detection wells of a fluidic chamber may have one or more of a variety of different volumes as needed for a given detection method. In some embodiments, individual detection wells may have any volume between about 0 and about 100 μL, e.g., about 1 μL, 2 μL, 3 μL, 4 μL, 5 μL, 6 μL, 7 μL, 8 μL, 9 μL, 10 μL, 11 μL, 12 μL, 13 μL, 14 μL, 15 μL, 16 μL, 17 μL, 18 μL, 19 μL, 20 μL, 21 μL, 22 μL, 23 μL, 24 μL, 25 μL, 26 μL, 27 μL, 28 μL, 29 μL, 30 μL, 31 μL, 32 μL, 33 μL, 34 μL, 35 μL, 36 μL, 37 μL, 38 μL, 39 μL, 40 μL, 41 μL, 42 μL, 43 μL, 44 μL, 45 μL, 46 μL, 47 μL, 48 μL, 49 μL, 50 μL, 51 μL, 52 μL, 53 μL, 54 μL, 55 μL, 56 μL, 57 μL, 58 μL, 59 μL, 60 μL, 61 μL, 62 μL, 63 μL, 64 μL, 65 μL, 66 μL, 67 μL, 68 μL, 69 μL, 70 μL, 71 μL, 72 μL, 73 μL, 74 μL, 75 μL, 76 μL, 77 μL, 78 μL, 79 μL, 80 μL, 81 μL, 82 μL, 83 μL, 84 μL, 85 μL, 86 μL, 87 μL, 88 μL, 89 μL, 90 μL, 91 μL, 92 μL, 93 μL, 94 μL, 95 μL, 96 μL, 97 μL, 98 μL, 99 μL, or about 100 μL, or any volume therebetween.

The number of detection wells available in a detection chamber 216 can be determined based on the number of biomarkers to be detected. The detection chamber comprises at least one detection well. In some embodiments, the detection chamber comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, or more than 200 detection wells. The presence of many detection wells in the detection chamber allows for high-throughput analysis of many genes. The presence of many detection wells in the detection chamber allows for multiplex analysis of many genes. However, in some embodiments, not all the detection wells may be used if there are fewer biomarkers to be measured compared to detection wells. In some embodiments, some detection wells of a fluidic device may be unused. In some embodiments, at least two of the detection wells may be configured to detect the same biomarker and used as replicates. In some cases, not all of the biomarkers are used to diagnose the disease or condition; some detection wells may be used as a housekeeping gene or biomarker to ensure the quality of the amplification reaction or the detection reaction.

An embodiment, variation, or example of the fluidic device 100 can thus be configured to detect a plurality of genes. In some embodiments, the fluidic device 100 is configured to detect at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, or more than 200 unique genes or biomarker genes. The fluidic device 100 can be configured to detect a plurality of genes in a high-multiplex and/or high-throughput manner.

Each detection well may have a minimum amount of nucleic acid that is sufficient to detect the desired plurality of genes. In some embodiments, the amount of nucleic acid in each individual detection well is between about 0.1 ng to about 100 ng, e.g., about 0.1 ng, 0.2 ng, 0.3 ng, 0.4 ng, 0.5 ng, 0.6 ng, 0.7 ng, 0.8 ng, 0.9 ng, 1.0 ng, 2.0 ng, 3.0 ng, 4.0 ng, 5.0 ng, 6.0 ng, 7.0 ng, 8.0 ng, 9.0 ng, 10.0 ng, 11.0 ng, 12.0 ng, 13.0 ng, 14.0 ng, 15.0 ng, 16.0 ng, 17.0 ng, 18.0 ng, 19.0 ng, 20.0 ng, 21.0 ng, 22.0 ng, 23.0 ng, 24.0 ng, 25.0 ng, 26.0 ng, 27.0 ng, 28.0 ng, 29.0 ng, 30.0 ng, 31.0 ng, 32.0 ng, 33.0 ng, 34.0 ng, 35.0 ng, 36.0 ng, 37.0 ng, 38.0 ng, 39.0 ng, 40.0 ng, 41.0 ng, 42.0 ng, 43.0 ng, 44.0 ng, 45.0 ng, 46.0 ng, 47.0 ng, 48.0 ng, 49.0 ng, 50.0 ng, 51.0 ng, 52.0 ng, 53.0 ng, 54.0 ng, 55.0 ng, 56.0 ng, 57.0 ng, 58.0 ng, 59.0 ng, 60.o ng, 61.0 ng, 62.0 ng, 63.0 ng, 64.0 ng, 65.0 ng, 66.0 ng, 67.0 ng, 68.0 ng, 69.0 ng, 70.0 ng, 71.0 ng, 72.0 ng, 73.0 ng, 74.0 ng, 75.0 ng, 76.0 ng, 77.0 ng, 78.0 ng, 78.0 ng, 79.0 ng, 80.o ng, 81.0 ng, 82.0 ng, 83.0 ng, 84.0 ng, 85.0 ng, 86.0 ng, 87.0 ng, 88.0 ng, 89.0 ng, 90.0 ng, 91.0 ng, 92.0 ng, 93.0 ng, 94.0 ng, 95.0 ng, 96.0 ng, 97.0 ng, 98.0 ng, 99.0 ng, or about 100.0 ng, or any amount therebetween.

In some embodiments, the detection chamber 216 can be coupled to a temperature control system 340. The temperature control unit can be used to heat the detection chamber 216 to one or more desired temperatures to carry out the detection reaction, for example an amplification reaction. In some embodiments, the temperature control system 340 may be located inside the instrument. Where the detection of mRNA is desired, the temperature control unit may be configured to maintain the detection chamber 216 at one or more desired temperatures for reverse transcription of mRNA to cDNA and amplification of cDNA. In some embodiments, the amplification may comprise an isothermal amplification and the detection chamber is kept roughly at a constant temperature during the amplification process. In some embodiments, the amplification may comprise loop-mediated isothermal amplification (LAMP). In some embodiments, the temperature control unit may be configured to maintain the detection chamber at any temperature between about 55° C. and about 75° C., e.g., about 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., or about 75° C., or any temperature therebetween.

The temperature control system 340 can maintain the detection chamber 216 at any given temperature for the desired amount of time needed to carry out detection of biomarkers. In some embodiments, the certain period of time may be between about 0 min and 60 min, e.g., about 0.5 min, 1 min, 1.5 min, 2 min, 2.5 min, 3 min, 3.5 min, 4 min, 4.5 min, 5 min, 5.5 min, 6 min, 6.5 min, 7 min, 7.5 min, 8 min, 8.5 min, 9 min, 9.5 min, 10 min, 10.5 min, 11 min, 11.5 min, 12 min, 12.5 min, 13 min, 13.5 min, 14 min, 14.5 min, 15 min, 15.5 min, 16 min, 16.5 min, 17 min, 17.5 min, 18 min, 18.5 min, 19 min, 19.5 min, 20 min, 20.5 min, 21 min, 21.5 min, 22 min, 22.5 min, 23 min, 23.5 min, 24 min, 24.5 min, 25 min, 25.5 min, 26 min, 26.5 min, 27 min, 27.5 min, 28 min, 28.5 min, 29 min, 29.5 min, 30 min, 30.5 min, 31 min, 31.5 min, 32 min, 32.5 min, 33 min, 33.5 min, 34 min, 34.5 min, 35 min, 35.5 min, 36 min, 36.5 min, 37 min, 37.5 min, 38 min, 38.5 min, 39 min, 39.5 min, 40 min, 40.5 min, 41 min, 41.5 min, 42 min, 42.5 min, 43 min, 43.5 min, 44 min, 44.5 min, 45 min, 45.5 min, 46 min, 46.5 min, 47 min, 47.5 min, 48 min, 48.5 min, 49 min, 49.5 min, 50 min, 50.5 min, 51 min, 51.5 min, 52 min, 52.5 min, 53 min, 53.5 min, 54 min, 54.5 min, 55 min, 55.5 min, 56 min, 56.5 min, 57 min, 57.5 min, 58 min, 58.5 min, 59 min, 59.5 min, or about 60 min, or about any time therebetween. In some embodiments, the fluidic device detects a plurality of genes or biomarker genes in a rapid manner.

In some embodiments, the product solution in the detection wells can contain a fluorescent moiety, which can be detected by the instrument 300. The fluorescent moiety can yield a fluorescence signal that is quantifiable and useful in measuring biomarker expression levels. In some embodiments, the fluorescent moiety may comprise one or more different (distinct) fluorescent labels attached to nucleotides or nucleotide analogs incorporated during nucleic acid amplification, e.g., isothermal amplification of a nucleic acid (e.g., 5-FAM (522 nm), ROX (608 nm), FITC (518 nm) and Nile Red (628 nm). In some embodiments, the fluorescent moiety may comprise a fluorescently tagged nucleic acid. In some embodiments, the fluorescent moiety may comprise a fluorescently tagged cDNA. In some embodiments, the fluorescence intensity of the fluorescent moiety may be correlated quantitatively with the amount of an mRNA and/or corresponding cDNA of a gene that is amplified in one or more detection wells. In some embodiments, the fluorescence signal of the fluorescent moiety may be quantified to measure the expression level of a gene. In some embodiments, the fluorescence signal of the fluorescent moiety may be quantified to measure the mRNA expression level of a gene. In some embodiments, quantitative real-time isothermal amplification of a target nucleic acid in a sample can be determined by detection of a single fluorophore species (e.g., ROX (608 nm)) attached to nucleotides or nucleotide analogs incorporated during isothermal amplification of the target nucleic acid. In some embodiments, each fluorophore species used emits a fluorescent signal that is distinct from any other fluorophore species, such that each fluorophore can be readily detected among other fluorophore species present in the assay.

Fluorescent moieties implemented can be associated with chemical families including: acridine derivatives, arylmethine derivatives, fluorescein derivatives, anthracene derivatives, tetrapyrrole derivatives, xanthene derivatives, oxazine derivatives, dipyrromethene derivatives, cyanine derivatives, squaraine derivates, squaraine rotaxane derivatives, naphthalene derivatives, coumarin derivatives, oxadiazole derivatives, pyrene derivatives, and/or other chemicals. Such fluorophores can further be attached to other functional groups as needed for tagging of biomarkers in a detectable manner.

In examples, fluorescent moieties implemented (e.g., for tagging of RNAs, DNAs, oligonucleotides, etc.) can include one or more of: FAM, (e.g., 6-FAM), Cy3™, Cy5™, Cy5.5™, TAMRA™ (e.g., 5-TAMRA, 6-TAMRA, etc.), MAX, JOE, TET™, ROX, TYE™ (e.g., TYE 563, TYE 665, TYE 705, etc.), Yakima Yellow®, HEX, TEX (e.g., TEX 615), SUN, ATTO™ (e.g., ATTO 488, ATTO 532, ATTO 550, ATTO 565, ATTO Rho101, ATTO 590, ATTO 633, ATTO 647, etc.), Alexa Fluor® (e.g., Alexa Fluor 488, Alexa Fluor 532, Alexa Fluor 546, Alexa Fluor 594, Alexa Fluor 647, Alexa Fluor 660, Alexa Fluor 750, etc.), IRDyes® (e.g., 5′IRDye 700, 5′IRDye 800, 5′IRDye 800CW, etc.), Rhodamine (e.g., Rhodamine Green, Rhodamine Red, Texas Red®, Lightcycler®, Dy 750, Hoechst dyes, DAPI dyes, SYTOX dyes, chromomycin dyes, mithramycin dyes, YOYO dyes, ethidium bromide dyes, acridine orange dyes, TOTO dytes, thiazole dyzes, CyTRAK dyes, propidium iodide dyes, LDS dyes, BODIPY dyes, and/or other dyes. In examples, fluorescent proteins for tagging of biomarkers and detection can include one or more of: cerulean, mCFP, mTurquoise, T-Sapphire, CyPet, ECFP, CFP, EBFP, Azurite, and/or other fluorescent proteins.

In some embodiments, methods of detecting amplification of a target nucleic acid in a sample in the detection wells and detection chamber by quantitative real-time isothermal amplification can include using intercalating fluorescent dyes, such as SYTO dyes (SYTO 9 or SYTO 82). In some embodiments, methods of detecting amplification of a target nucleic acid in a sample in the detection wells and detection chamber by quantitative real-time isothermal amplification can include using unlabeled primers to isothermally amplify the target nucleic acid in the sample, and a labeled probe (e.g., having a fluorophore) to detect isothermal amplification of the target nucleic acid in the sample. In some embodiments, unlabeled primers are used to isothermally amplify a target nucleic acid present in the sample, and a probe is used having a 5-FAM dye label on the 5′ end and a minor groove binder (MGB) and non-fluorescent quencher on the 3′ end to detect isothermal amplification of the target nucleic acid (e.g., TaqMan Gene Expression Assays from ThermoFisher Scientific).

The instrument 300 can include a detection system 350 for detection of the fluorescence of the fluorescent moiety(ies) in the detection wells. The detection system 350 can include a fluorometer, a spectrofluorometer, or an imaging system. In some embodiments, the fluorescence is detected by an imaging system coupled to the instrument 300. In some embodiments, the imaging system may be coupled to the fluidic device 100. In some embodiments, the detection system 350 can acquire an image of one or more of the detection wells. An image of the one or more detection wells acquired by the imaging system may be processed by a processing subsystem 360 comprising software architecture, which can be configured to quantify the fluorescence signal in the one or more detection wells. In some embodiments, the processing subsystem 360 can be coupled to the instrument 300. The fluorescence intensity in the image of individual detection wells may be correlated with the mRNA expression level of a biomarker gene. In some embodiments, the fluorescence signal in the image may be processed to measure the mRNA expression level of a gene. In some embodiments, the biomarker gene may be a host response biomarker. In some embodiments, the biomarker gene may comprise a housekeeping gene which is not expected to show significant variation in expression level and is used to confirm that the detection reaction is successful. In some embodiments, the biomarker gene may comprise an internal control nucleic acid sequence. In some embodiments, the processing subsystem 360 can comprise architecture for normalizing the expression level of a biomarker to the expression level of a housekeeping gene or a control nucleic acid.

The processing subsystem 360 can include a plurality of units useful for quantifying the fluorescence signal in one or more detection wells. In some embodiments, the software module may comprise a pre-processing unit and a quantification unit. In some embodiments, the software module may comprise a machine learning classifier. In some embodiments, the pre-processing unit of the software module may convert the fluorescence signal in an image to a vector indicative of the expression level of a biomarker and feed the vector to the quantification unit. In some embodiments, the vector may comprise a plurality of quantitative features. In some embodiments, the machine learning classifier may calculate a composite biomarker, also referred to as a biomarker score or a score, based on the vector. The composite biomarker or biomarker score may integrate expression levels of a plurality of biomarker genes. In some cases, the plurality of biomarker genes comprise both over-expressed and under-expressed genes and is a composite of the biomarker genes. The composite biomarker can be useful in diagnosing a disease, disorder, or condition in a subject from which the biological sample is obtained.

In some embodiments, one or more of the modules of the processing subsystem 360 can be stored externally on the cloud. For example, if the machine learning classifier is stored externally on the cloud, the software module may upload the vector to the cloud where the machine learning classifier is stored. After the machine learning classifier determines the score (or composite biomarker) based on the uploaded vector, the score (or composite biomarker) is sent back to the software module where the result can be outputted to a user. In another example, if the pre-processing unit and the quantification unit are also stored externally on the cloud, the software module may upload one or more images of the one or more detection wells to the cloud where the pre-processing unit and the quantification unit are stored. After converting the fluorescence signal in the one or more images to a vector indicative of the expression level of a biomarker and quantification, the vector can then be fed to the machine learning classifier which calculates the score. The score can then be sent back to the software module where the result can be outputted to the user. The fluidic device 100 can be configured to detect a plurality of genes or biomarker genes in a quantitative manner.

The fluidic device 100 and instrument 300 can achieve complete analysis of a biological sample by disclosed herein within 20 minutes, 21 minutes, 22 minutes, 23 minutes, 24 minutes, 25 minutes, 26 minutes, 27 minutes, 28 minutes, 29 minutes, 30 minutes, 31 minutes, 32 minutes, 33 minutes, 34 minutes, 35 minutes, 36 minutes, 37 minutes, 38 minutes, 39 minutes, 40 minutes, 41 minutes, 42 minutes, 43 minutes, 44 minutes, 45 minutes, 46 minutes, 47 minutes, 48 minutes, 49 minutes, 50 minutes, 51 minutes, 52 minutes, 53 minutes, 54 minutes, 55 minutes, 56 minutes, 57 minutes, 58 minutes, 59 minutes, or about 60 minutes, or any time therebetween.

Completing the analysis of a biological sample refers to all the steps performed on the fluidic device 100 and/or instrument 300 to yield the diagnosis output. For example, this includes the steps of nucleic acid extraction to yield one or more nucleic acids as well as one or more detection reactions, such as nucleic acid amplification, to determine biomarker expression levels. In some embodiments, it may take less than 30 min to complete the analysis of a biological sample by the fluidic device disclosed herein and/or the instrument disclosed herein. In some embodiments, it may take less than 15 min to complete the analysis of a biological sample using the fluidic device disclosed herein and/or the instrument disclosed herein. In some embodiments, it may take any time between about 0 min and about 60 min to complete the analysis of a biological sample by the fluidic device disclosed herein and/or the instrument disclosed herein, e.g., about 1 min, 2 min, 3 min, 4 min, 5 min, 6 min, 7 min, 8 min, 9 min, 10 min, 11 min, 12 min, 13 min, 14 min, 15 min, 16 min, 17 min, 18 min, 19 min, 20 min, 21 min, 22 min, 23 min, 24 min, 25 min, 26 min, 27 min, 28 min, 29 min, 30 min, 31 min, 32 min, 33 min, 34 min, 35 min, 36 min, 37 min, 38 min, 39 min, 40 min, 41 min, 42 min, 43 min, 44 min, 45 min, 46 min, 47 min, 48 min, 49 min, 50 min, 51 min, 52 min, 53 min, 54 min, 55 min, 56 min, 57 min, 58 min, 59 min, or about 60 min, any time therebetween.

In examples, the fluidic device 100 can be structured to perform (e.g., optimized for) sample processing and characterization based upon a first set of informative biomarker components (i.e., informative genes), a second set of housekeeping biomarker components (e.g., housekeeping genes), and a third set of control components (e.g., control genes). Detection and analysis of the first set of informative biomarker components (i.e., informative genes) provides information regarding the condition of the subject (where exemplary conditions are described herein), the second set of housekeeping biomarker components (e.g., housekeeping genes) provides control from subject-to-subject, and the third set of control biomarker components (e.g., control genes) are indicative of proper functioning of the fluidic device (e.g. in relation to cartridge-to-cartridge consistency and reproducibility of analyses), with respect to proper sample preparation and proper amplification, respectively. The third set of control biomarker components is also used to normalize/provide corrections for characterizations (e.g., abundance characterizations) of other biomarker components.

In relation to the fluidic device 100 being structured for sample processing and characterization, as described, the selection of the first set of informative biomarkers, the second set of housekeeping biomarkers, and the third set of control biomarkers is based upon an assessment of biomarker targets that amplify suitably (e.g., using a LAMP assay), using the reaction chamber(s) and/or detection chamber(s) of the fluidic device 100, in coordination with instrument aspects described below. Upon processing the biological sample and analyzing signals related to the first set of informative biomarkers, the second set of housekeeping biomarkers, and the third set of control biomarkers, the system and method are structured to return a characterization of the biological sample based upon a composite biomarker.

According to a specific example, the first set of informative biomarkers includes the following genes: ANKRD22, ARG1, BATF, C3AR1, CD163, CEACAM1, CLEC5A, CTSL1, DEFA4, HERC5, HLA-DMB, IFI27, IFI44, IFI44L, IL18R1, IL1R2, ISG15, JUPv9, KCNJ2, LY86, OASL, OLFM4, PSMB9, RSAD2_PT4, S100A12_PT1, TDRD9_PT3, TGFBI, XAF1_PT4, and ZDHHC19

According to a specific example, the second set of housekeeping biomarkers includes the following genes: KPNA6, RREB1, and YWHAB.

According to a specific example, the third set of control biomarkers includes RNA controls (e.g., ERCC17, ERCC59, additional or alternative control biomarkers, etc.).

A fluidic device 100 of the disclosure, as provided in the various aspects and embodiments above, can be configured to detect a plurality of genes (e.g., biomarker genes) in a rapid, high-multiplex/high-throughput, and quantitative manner. The detected expression levels of genes or biomarker genes using a fluidic device of the disclosure can be useful in informing on a disease, disorder, or condition of a patient.

### 2.4 System—Instrument

Embodiments, variations, and examples of the system 10 can further include an instrument 300, as shown in FIGS. 1 and 3. In some embodiments, the instrument 300 accepts a fluidic device disclosed herein and can be used for measuring gene expression from a biological sample. In some embodiments, the instrument 300 is used in conjunction with a fluidic device 100 disclosed herein to measure gene expression from a biological sample. Such measurements can be useful in diagnosis and/or treatment of a subject with a disease, disorder, or condition. In some embodiments, a system comprising a fluidic device and instrument as disclosed herein may be useful for a rapid point-of-care transcriptomics test that measures the expression level of biomarkers. In some embodiments, the biomarkers are differentially expressed. In some embodiments, the biomarkers are related to an immune response.

In some embodiments, the instrument 300 includes a temperature control system 340, a mechanical actuator 370, a pneumatic assembly 380, a detection system 350, and a processing subsystem 360, all of which are coupled to the fluidic device.

The instrument 300 includes architecture for receiving a fluidic device and biological sample. After being loaded into the instrument 300, the temperature control system 340, mechanical actuator 370, pneumatic assembly 380, and detection system 350 interface with the fluidic device 100 to complete the analysis of the biological sample. In some embodiments, the mechanical actuator 370 can be used to compress a blister 106 storing a reagent in the reagent storage zone 214 of a fluidic device. In some embodiments, the pneumatic assembly can comprise a pump which operates a valve which opens and closes a channel of the fluidic device.

The pneumatic assembly 380 can be coupled to the fluidic device 100 through a pneumatic interface located on the fluidic device 100. The pneumatic interface can comprise a plurality of gas ports 110 controlled by one or more valves 112. In some embodiments, a silicon gasket 108 sits on top of the gas ports to ensure that a seal is formed. In some embodiments, the pneumatic interface may be coupled to the pneumatic assembly, which is configured to move a fluid inside the fluidic device through one or more channels. The pneumatic assembly, in some embodiments, may be activated to move fluid to the metering compartment and reaction compartments. The pneumatic assembly can also be used to flow the biological sample from the metering compartment through the reagent storage zone. The pneumatic assembly can also be used to move reagents from one or more blisters in the reagent storage zone to a reaction compartment. The pneumatic assembly can also be used to move extracted nucleic acids mixed with one or more reagents to the detection chamber where expression levels can be measured.

In some embodiments, the instrument 300 may further comprise a magnet module 390 capable of applying a magnetic field. The magnet module can be used, for example, during nucleic acid extraction. During nucleic acid extraction, nucleic acids may be tethered to magnetic beads, for example via hybridization to complementary nucleic acid sequences. For example, where extraction of mRNA from the biological sample is desired, the magnetic beads can be functionalized with oligo-dT which is capable of hybridizing to the polyA tail of mRNA. The magnet module can be used to immobilize magnetic beads during washing steps to remove components such as buffers originally used to stabilize the biological sample and cellular proteins that are not needed for nucleic acid analysis. After washing and isolating the desired nucleic acids for analysis, the nucleic acids can be eluted from the beads in an elution buffer and then transferred to another compartment of the device for further analysis. The magnet module can be used to immobilize the magnetic beads so that they do not interfere with downstream steps.

In some embodiments, the instrument 300 can include a temperature control system 340. The temperature control system 340 can be used to modulate the temperature of one or more components of a fluidic device 100. For example, the temperature control system 340 can be used to maintain the reaction chamber 208 at a desired reaction chamber temperature, for example for nucleic acid extraction. The temperature control system 340 can also be used to maintain the detection chamber 216 at a desired detection chamber temperature for measuring expression levels of biomarkers, for example amplification reactions for determining mRNA expression levels of biomarker genes.

In some embodiments, the instrument may comprise a detection system 350. The detection system 350 can be utilized to capture one or more images of the detection wells during an amplification process. For example, images of the one or more detection wells may be captured at regular intervals during amplification. Changes in fluorescence intensity over time as amplification progresses can then be quantified to determine expression levels of biomarkers in the biological sample.

In some embodiments, the instrument 300 can further include a processing subsystem 360 comprising software architecture. The detection system 350 can include a plurality of units useful for quantifying the fluorescence signal in one or more detection wells. The detection system 350 can include a pre-processing unit and a quantification unit. The software module can also comprise a machine learning classifier which calculates a composite biomarker, also referred to as a biomarker score or a score, from the measured expression levels of the biomarker genes.

Embodiments, variations, and examples of the instrument 300 can further have functionality for (e.g., with the processing subsystem 360 and other subsystems/assemblies described) generating and/or provide notifications regarding quality of sample drawing (e.g., if a blood draw was suitable or unsuitable, if sample metering at the fluidic device was performed correctly, etc.). Embodiments, variations, and examples of the instrument 300 can additionally or alternatively have functionalized for generating and/or providing notifications regarding any prior use of the fluidic device 100 (e.g., for cartridges or other disposable components that should not be used multiple times). Identification that a fluidic device 100 has been previously used can be determined upon scanning a barcode associated with the fluidic device (e.g., barcode used to label a cartridge), where scanning the barcode can indicate a history of usage of the fluidic device. Scanning components of the instrument 300 can include scanning elements (e.g., optical scanning elements, near-field communication elements, radiofrequency identification (RFID) elements, etc.) for determining usage history of a fluidic device unit. As such, scanning elements of the instrument 300 can generate data that can be processed by the processing subsystem 360 to assess prior unit of a fluidic device 100 unit being interfaced with the instrument 300, where scanning the barcode can indicate a history of usage of the fluidic device.

The instrument 300 and/or fluidic device 100 can further include one or more sensors for detecting fluid and/or evidence of fluid or other sample processing components within the fluidic device 100, such that identification that the fluidic device 100 has been previously used can be determined upon sensing presence of fluid (or evidence of fluid) within components (e.g., chambers, channels, wells, etc.). Exemplary sensors can include one or more capacitive sensors, optical sensors, temperature sensors, or other suitable sensors.

### 3. Method

As shown in FIG. 5, a method 500 for processing a biological sample of a subject is disclosed herein. In some embodiments, the method may be carried out by a fluidic device disclosed herein and/or an instrument disclosed herein. In some embodiments, the method may be used for a rapid point of care diagnosis or test of a subject with a disease, disorder, or condition. In some embodiments, the method may be used for a rapid point of care transcriptomics test that measures the RNA expression level of a biomarker gene which is differentially expressed in a disease, disorder, or condition. In some embodiments, multiple biomarkers are used in conjunction for the prognosis and/or diagnosis of a disease, disorder, or condition. Some of the multiple biomarkers may be increased in expression for the particular disease, disorder, or condition. Some of the multiple biomarkers may be decreased in expression. In some cases, such a method can be useful in the prognosis and/or diagnosis of infections such as bacterial infection, viral infection, fungal infection, severe infection, or combinations thereof. In some embodiments, the biomarker may be related to an immune response. In some embodiments, the method disclosed herein can be carried out entirely and solely by the fluidic device and/or instrument once a biological sample is loaded into the fluidic device and/or instrument. In some embodiments, the method disclosed herein does not require any additional sample preparation. In some embodiments, the method disclosed herein does not require any other processing of a biological sample, such as nucleic acid extraction, by a user. In some embodiments, the method disclosed herein may measure multiplex genes or biomarkers.

In some embodiments, one or more steps of the method 500 disclosed herein may be carried out at a healthcare facility including, but not limited to, a hospital, a clinic, or a pharmacy. In some embodiments, one or more steps of the method may be carried out at a laboratory. In some embodiments, one or more steps of the method may be carried out by a healthcare professional including, but not limited to, a doctor, a nurse, a pharmacist, or a laboratory technician.

In particular, embodiments, variations, and examples of the method 500 achieve unprecedented performance in runtime, sample volume required to return characterizations of a biological sample, and other performance aspects described, based upon the following factors and other factors, including:

Omission of performance of deoxyribonucleic acid (DNA) degradation involving a Deoxyribonuclease (DNase), with omission of amplification of genomic DNA. As such, the method can be performed with elimination of steps involving DNase use, as attributed to primer design factors described herein.

Combination of the biological sample with an intracellular RNA stabilization solution (e.g., PAXgene™) for biological samples including intracellular ribonucleic acids (RNAs), given that lysis has already occurred prior to introduction to the fluidic device.

Implementation of a significant reduction in number of wash steps, by executing a LAMP assay with tolerance to residual inhibitors. In examples, the number of wash steps can be less than 10 wash steps, less than 9 wash steps, less than 8 wash steps, less than 7 wash steps, less than 6 wash steps, less than 6 wash steps, less than 5 wash steps, less than 4 wash steps, less than 3 wash steps, or less than 2 wash steps.

Application of active drying (e.g., forced convection drying) of the sample processing reagents within the fluidic device, to achieve significant reductions in sample processing time.

Performance of fluid agitation (e.g., pneumatic-driven fluid agitation) and homogenization of functionalized particles, with bubble mixing, within the fluidic device, thereby producing rapid binding of RNAs of the biological sample to capture molecules within the fluidic device and significantly reducing runtime.

Performance of parallelization of sample processing steps, including but not limited to sample metering, reagent rehydration, heating of buffers, dispensing wet reagents, and other sample processing steps.

In some embodiments, the method 500 can include a step 502 of obtaining a biological sample from a subject. In some embodiments, obtaining a biological sample from a subject may be carried out at a laboratory or in some cases can be carried out at a healthcare facility including, but not limited to, a hospital, a clinic, or a pharmacy. In some embodiments, the biological sample may be obtained by a healthcare professional including, but not limited to, a doctor, a nurse, a pharmacist, or a laboratory technician. In some embodiments, obtaining the biological sample can include loading the biological sample into a sample container. In some embodiments, the sample container may comprise a vial, a tube, a bottle, or a cup. In some embodiments, the sample container may be a PAXgene® Blood RNA Tube, Tempus Blood RNA Tube, or any other commercially available container capable of stabilizing RNA. In some embodiments, the sample container cannot be opened, manipulated, or tampered with by a user before or after the biological sample is loaded into the sample container. In some embodiments, the biological sample is subject to processing before being loaded into the sample container. In some embodiments, the biological sample is not subject to processing before being loaded into the sample container. As such, the step of loading the biological sample can omit opening of the collection tube and can omit manual transfer (e.g., by pipetting, etc.) of the biological sample from the collection tube into the fluidic device. In some embodiments, the biological sample is not subject to manipulation or tampering by a user before or after the biological sample is loaded into the sample container. In some embodiments, the biological sample may be a biological specimen obtained from a subject. In some embodiments, the biological sample may be in solid form. In some embodiments, the biological sample may be in liquid form. In some embodiments, the biological sample may be converted from a solid form to a liquid form before being loading into the fluidic device. In some embodiments, the biological sample may comprise whole blood, a buffy coat, plasma, serum, saliva, urine, tissue biopsy, peripheral blood mononucleated cell (PBMC), a band cell, a neutrophil, a monocyte, a T cell, a nasal swab, or a combination thereof.

As disclosed above, in some embodiments, the method 500 can include a step 504 of loading/receiving the biological sample into a fluidic device disclosed herein. In some embodiments, the step 504 can include inserting the sample container 102 containing the biological sample into the sample holder 104 of a fluidic device. In some embodiments, the sample container may be removable from the sample holder of the fluidic device. In some embodiments, the biological sample is subject to processing after being loaded into the sample container but before the sample container is inserted into the fluid device. In some embodiments, the biological sample is not subject to processing after being loaded into the sample container but before the sample container is inserted into the fluid device.

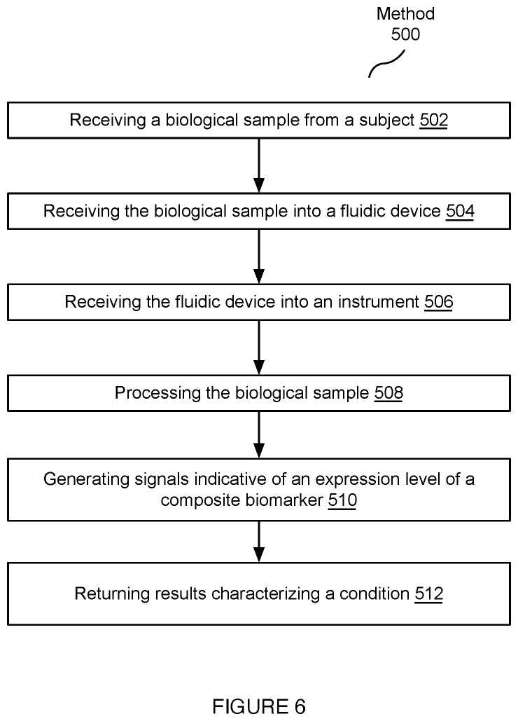

In the example shown in FIGS. 5A and 6, receiving the sample container at the fluidic device can include providing, at the fluidic device, a valve configured in a normally-closed operation mode, where receiving the sample container in Step 504 includes providing increased resistance to opening the valve with the collection tube by: providing the valve with a valve seat surrounded by a recess for an elastomeric membrane, wherein the valve seat is one of: a) flush with and b) raised beyond the elastomeric membrane. The recess is provided for fluid to move through, and the membrane seals the top of the valve seat. When the membrane is open, fluid moves through the recess 384/channel. The normally-closed valve can be positioned at an interface between the sample container and the fluidic device 100 (e.g., cartridge), in order to provide control over fluid delivery from the sample container into the fluidic device (e.g., when a user docks the collection tube at the fluidic device 100/cartridge), such that processing the sample can be performed with greater control (e.g., upon inserting the fluidic device 100 into the instrument 300).

In relation to Step 504, the valve can further be structured to provide increased resistance to opening of the valve when a sample container is inserted into the holder of the fluidic device (e.g., upon interfacing the collection tube with the fluidic device), by laser welding (or otherwise coupling) the membrane to the valve seat in a manner that increases resistance to opening, such that opening the valve occurs at a threshold cracking pressure. As such Step 504 can include providing increased resistance to opening the valve with the collection tube/sample container, by bonding (e.g., laser welding) the elastomeric membrane to the valve seat.

In relation to welding/coupling the membrane to the valve seat, the valve 385 can be structured with a weld (e.g., laser weld, thermal bond, other bond, etc.) at one side of the valve seat, where the valve stays in the normally-closed operation mode until a cracking pressure is exceeded upon coupling the collection tube with the fluidic device 100. Providing a radial weld can include providing a weld that spans approximately 5 degrees, 10 degrees, 15 degrees, 20 degrees, 25 degrees, 30 degrees, 35 degrees, 40 degrees, 45 degrees, 50 degrees, 55 degrees, 60 degrees, 65 degrees, 70 degrees, 75 degrees, 80 degrees, 85 degrees, 90 degrees, 95 degrees, 100 degrees, 105 degrees, 110 degrees, 115 degrees, 120 degrees, 125 degrees, 130 degrees, 135 degrees, 140 degrees, 145 degrees, 150 degrees, 155 degrees, 160 degrees, 165 degrees, 170 degrees, 175 degrees, 180 degrees, 185 degrees, 190 degrees, 195 degrees, 200 degrees, 205 degrees, 210 degrees, 215 degrees, 220 degrees, 225 degrees, 230 degrees, 235 degrees, 240 degrees, 245 degrees, 250 degrees, 255 degrees, 260 degrees, 265 degrees, 270 degrees, 275 degrees, 280 degrees, 285 degrees, 290 degrees, 295 degrees, 300 degrees, 305 degrees, 310 degrees, 315 degrees, 320 degrees, 325 degrees, 330 degrees, 335 degrees, 340 degrees, 345 degrees, 350 degrees, 355 degrees, 360 degrees or an intermediate number of degrees. Radial welds can be continuous, or intermittently positioned about the valve seat 386.

As shown in FIG. 5B, providing a weld in relation to Step 504 can include providing the a weld that takes the form of one or more spot/line welds, where the spot/line welds have a length of approximately 0.05 mm, 0.1 mm, 0.15 mm, 0.2 mm, 0.25 mm, 0.3 mm, 0.35 mm, 0.4 mm, 0.45 mm, 0.5 mm, 0.55 mm, 0.6 mm, 0.65 mm, 0.7 mm, 0.75 mm, 0.8 mm, 0.85 mm, 0.9 mm, 0.95 mm, 1 mm, or another suitable length. A specific example of the weld 388 includes a 0.2 mm line/spot weld, on the valve seat 386 on one side of the seat, which increases the fluidic resistance so that the valve stays closed until a threshold cracking pressure is exceeded.

In examples of Step 504, the threshold cracking pressure provided by the normally-closed valve can be approximately 5 kPa, 7.5 kPa, 10 kPa, 15 kPa, 20 kPa, 25 kPa, 30 kPa, 35 kPa, 40 kPa, 45 kPa, 50 kPa, 55 kPa, 60 kPa, 65 kPa, 70 kPa, 75 kPa, 80 kPa, 85 kPa, 90 kPa, 95 kPa, 100 kPa, an intermediate cracking pressure, or a greater cracking pressure. A specific example of the weld 388 provides a threshold cracking pressure of approximately 20 kPa.

In relation to Step 504, the method 500 can include providing increased resistance to opening the valve with the collection tube 102 by providing the weld 388 with an offset to an opening into the valve less than 1.5 millimeters, less than 1 millimeter, less than 0.9 millimeters, less than 0.8 millimeters, less than 0.7 millimeters, less than 0.6 millimeters, less than 0.5 millimeters, less than 0.4 millimeters, less than 0.3 millimeters, less than 0.2 millimeters, or less. In a specific example, the offset of the weld position to the valve (e.g., opening) is less than 0.3 mm.

As disclosed above, in some embodiments, the method 500 can include a step 506 of receiving the fluidic device disclosed herein into an instrument disclosed herein. In some embodiments, the fluidic device may be a single use device. In some embodiments, the step 506 may comprise selecting a computer program installed on the instrument through the display of the instrument. In some embodiments, the computer program may direct the instrument and fluidic device to carry out one or more tests selected for the prognosis and/or diagnosis of a disease, disorder, or condition. In some embodiments, the step 506 may comprise running the computer program on the instrument to carry out the method 500.

As disclosed above, in some embodiments, the method 500 can include a step 508 of processing the biological sample using the fluidic device disclosed herein and/or the instrument disclosed herein. Processing the biological sample, for example, can comprise extracting nucleic acid from the biological sample inside the reaction chamber of the fluidic device using a plurality of reagents that are stored on the fluidic device, for example reagents stored within a blister or the reagent storage zone. In some embodiments, processing the biological sample can comprise a cell lysis step to ensure that the cells are lysed before nucleic acid extraction. In some embodiments, the nucleic acid to be extracted comprises RNA, such as an mRNA or a miRNA. If analysis of DNA is desired, the method can also comprise extraction of DNA. In some embodiments where analysis of mRNA is desired, mRNA is extracted from the biological sample within the fluidic device. In some cases, both DNA and RNA can be extracted from the biological sample but only the DNA or RNA is analyzed in downstream steps. Once the nucleic acid extraction is complete, a solution containing purified nucleic acid may then be available for the next step of analysis.

The extracted nucleic acid can be transferred to another location on the fluidic device for further analysis. The amount of extracted nucleic acid that is extracted for additional analysis may depend on the number of biomarkers desired to be amplified. For example, if there are more biomarkers that are desired to be analyzed, more nucleic acid would be needed compared to if fewer biomarkers were to be analyzed. In some embodiments, the extracted nucleic acid for further analysis is between about 0.1 ng/L and about 10.0 ng/L, e.g., about 0.1 ng/L, 0.2 ng/L, 0.3 ng/L, 0.4 ng/L, 0.5 ng/L, 0.6 ng/L, 0.07 ng/L, 0.8 ng/L, 0.9 ng/L, 1.0 ng/L, 1.1 ng/L, 1.2 ng/L, 1.3 ng/L, 1.4 ng/L, 1.5 ng/L, 1.6 ng/L, 1.7 ng/L, 1.8 ng/L, 1.9 ng/L, 2.0 ng/L, 2.1 ng/L, 2.2 ng/L, 2.3 ng/L, 2.4 ng/L, 2.5 ng/L, 2.6 ng/L, 2.7 ng/L, 2.8 ng/L, 2.9 ng/L, 3.0 ng/L, 3.1 ng/L, 3.2 ng/L, 3.3 ng/L, 3.4 ng/L, 3.5 ng/L, 3.6 ng/L, 3.7 ng/L, 3.8 ng/L, 3.9 ng/L, 4.0 ng/L, 4.1 ng/L, 4.2 ng/L, 4.3 ng/L, 4.4 ng/L, 4.5 ng/L, 4.6 ng/L, 4.7 ng/L, 4.8 ng/L, 4.9 ng/L, 5.0 ng/L, 5.1 ng/L, 5.2 ng/L, 5.3 ng/L, 5.4 ng/L, 5.5 ng/L, 5.6 ng/L, 5.7 ng/L, 5.8 ng/L, 5.9 ng/L, 6.0 ng/L, 6.1 ng/L, 6.2 ng/L, 6.3 ng/L, 6.4 ng/L, 6.5 ng/L, 6.6 ng/L, 6.7 ng/L, 6.8 ng/L, 6.9 ng/L, 7.0 ng/L, 7.1 ng/L, 7.2 ng/L, 7.3 ng/L, 7.4 ng/L, 7.5 ng/L, 7.6 ng/L, 7.7 ng/L, 7.8 ng/L, 7.9 ng/L, 8.0 ng/L, 8.1 ng/L, 8.2 ng/L, 8.3 ng/L, 8.4 ng/L, 8.5 ng/L, 8.6 ng/L, 8.7 ng/L, 8.8 ng/L, 8.9 ng/L, 9.0 ng/L, 9.1 ng/L, 9.2 ng/L, 9.3 ng/L, 9.4 ng/L, 9.5 ng/L, 9.6 ng/L, 9.7 ng/L, 9.8 ng/L, 9.9 ng/L, or about 10.0 ng/L, or any amount therebetween. In some embodiments, the amount of extracted nucleic acid for further analysis is between about 10 ng/L and about 100 ng/L, e.g., about 10 ng/L, 11 ng/L, 12 ng/L, 13 ng/L, 14 ng/L, 15 ng/L, 16 ng/L, 17 ng/L, 18 ng/L, 19 ng/L, 20 ng/L, 21 ng/L, 22 ng/L, 23 ng/L, 24 ng/L, 25 ng/L, 26 ng/L, 27 ng/L, 28 ng/L, 29 ng/L, 30 ng/L, 31 ng/L, 32 ng/L, 33 ng/L, 34 ng/L, 35 ng/L, 36 ng/L, 37 ng/L, 38 ng/L, 39 ng/L, 40 ng/L, 41 ng/L, 42 ng/L, 43 ng/L, 44 ng/L, 45 ng/L, 46 ng/L, 47 ng/L, 48 ng/L, 49 ng/L, 50 ng/L, 51 ng/L, 52 ng/L, 53 ng/L, 54 ng/L, 55 ng/L, 56 ng/L, 57 ng/L, 58 ng/L, 59 ng/L, 60 ng/L, 61 ng/L, 62 ng/L, 63 ng/L, 64 ng/L, 65 ng/L, 66 ng/L, 67 ng/L, 68 ng/L, 69 ng/L, 70 ng/L, 71 ng/L, 72 ng/L, 73 ng/L, 74 ng/L, 75 ng/L, 76 ng/L, 77 ng/L, 78 ng/L, 79 ng/L, 80 ng/L, 81 ng/L, 82 ng/L, 83 ng/L, 84 ng/L, 85 ng/L, 86 ng/L, 87 ng/L, 88 ng/L, 89 ng/L, 90 ng/L, 91 ng/L, 92 ng/L, 93 ng/L, 94 ng/L, 95 ng/L, 96 ng/L, 97 ng/L, 98 ng/L, 99 ng/L, or about 100 ng/L, or any amount therebetween. In some embodiments, the extracted nucleic acid for further analysis is between about 100 ng/L and about 1 microgram/L (μg/L), e.g., about 100 ng/L and about 1 microgram/L (μg/L), e.g., about 100 ng/L, 110 ng/L, 120 ng/L, 130 ng/L, 140 ng/L, 150 ng/L, 160 ng/L, 170 ng/L, 180 ng/L, 190 ng/L, 200 ng/L, 210 ng/L, 220 ng/L, 230 ng/L, 240 ng/L, 250 ng/L, 260 ng/L, 270 ng/L, 280 ng/L, 290 ng/L, 300 ng/L, 310 ng/L, 320 ng/L, 330 ng/L, 340 ng/L, 350 ng/L, 360 ng/L, 370 ng/L, 380 ng/L, 390 ng/L, 400 ng/L, 410 ng/L, 420 ng/L, 430 ng/L, 440 ng/L, 450 ng/L, 460 ng/L, 470 ng/L, 480 ng/L, 490 ng/L, 500 ng/L, 510 ng/L, 520 ng/L, 530 ng/L, 540 ng/L, 550 ng/L, 560 ng/L, 570 ng/L, 580 ng/L, 590 ng/L, 600 ng/L, 610 ng/L, 620 ng/L, 630 ng/L, 640 ng/L, 650 ng/L, 660 ng/L, 670 ng/L, 680 ng/L, 690 ng/L, 700 ng/L, 710 ng/L, 720 ng/L, 730 ng/L, 740 ng/L, 750 ng/L, 760 ng/L, 770 ng/L, 780 ng/L, 790 ng/L, 800 ng/L, 810 ng/L, 820 ng/L, 830 ng/L, 840 ng/L, 850 ng/L, 860 ng/L, 870 ng/L, 880 ng/L, 890 ng/L, 900 ng/L, 910 ng/L, 920 ng/L, 930 ng/L, 940 ng/L, 950 ng/L, 960 ng/L, 970 ng/L, 980 ng/L, 990 ng/L, or about 1000 ng/L, or any amount therebetween. In some embodiments, the amount of nucleic acid disclosed above may be applicable to any type of nucleic acid including, but not limited to, a DNA, a cDNA, a RNA, a mRNA, and a miRNA.

As disclosed above, in some embodiments, measuring the expression levels of biomarkers comprises nucleic acid amplification. As previously described, any suitable amplification method can be employed. For example, to measure the expression level of a particular biomarker, the amount of mRNA corresponding to a biomarker gene is determined by amplifying the particular mRNA from the biological sample. In some embodiments, mRNA is first reverse transcribed to complementary DNA, or cDNA. The cDNA can then be amplified. In some embodiments, the amplification method is isothermal amplification. In some embodiments, the isothermal amplification may comprise a loop-mediated isothermal amplification (LAMP). In some embodiments, the isothermal amplification assay comprises a digital reverse-transcription loop-mediated isothermal amplification (dRT-LAMP) reaction for quantifying the target nucleic acid. Typically, LAMP assays produce a detectable signal (e.g., fluorescence) during the amplification reaction. In some embodiments, fluorescence can be detected and quantified. Any suitable method for detecting and quantifying florescence can be used.

In some embodiments, the amplification comprises polymerase chain reaction (PCR) amplification, LAMP amplification, recombinase polymerase amplification (RPA), ligase chain reaction, branched DNA amplification, nucleic acid sequence-based amplification (NASBA), strand displacement assay (SDA), transcription-mediated amplification, rolling circle amplification, helicase-dependent amplification (HDA), single primer isothermal amplification (SPIA), nicking and extension amplification reaction (NEAR), or transcription mediated assay (TMA).

In some embodiments, the nucleic acids extracted from the biological sample are subjected to a clustered regularly interspaced short palindromic repeats-Cas (CRISPR-Cas) reaction to determine the expression levels of nucleic acid biomarkers in the biological sample.

For an amplification reaction, the solution comprising extracted nucleic acid can be transported to the detection chamber of the fluidic device where one or more primers may be present in detection wells. In some embodiments, a plurality of primers are used to amplify a biomarker gene and is referred to as a primer set. In some embodiments, the primer or primer set selectively amplifies mRNA in a mixture of RNA and DNA. In some embodiments, the primer or primer set may be derived only from an exon region of a gene but not from an intron region of a gene. Where multiple biomarkers are analyzed in parallel, the primers or primer sets can be designed such that the amplification efficiencies are comparable between the multiple biomarkers and differences in amplification can be attributed to differences in the relative amounts of biomarker nucleic acid present in the biological sample.

In some embodiments, the concentration of the primer or primer set in a detection well may be between 0 μM and 10 μM, e.g., about 0.001 μM, 0.002 μM, 0.003 μM, 0.004 μM, 0.005 μM, 0.006 μM, 0.007 μM, 0.008 μM, 0.009 μM, 0.01 μM, 0.011 μM, 0.012 μM, 0.013 μM, 0.014 μM, 0.015 μM, 0.016 μM, 0.017 μM, 0.018 μM, 0.019 μM, 0.02 μM, 0.021 μM, 0.022 μM, 0.023 μM, 0.024 μM, 0.025 μM, 0.026 μM, 0.027 μM, 0.028 μM, 0.029 μM, 0.03 μM, 0.031 μM, 0.032 μM, 0.033 μM, 0.034 μM, 0.035 μM, 0.036 μM, 0.037 μM, 0.038 μM, 0.039 μM, 0.04 μM, 0.041 μM, 0.042 μM, 0.043 μM, 0.044 μM, 0.045 μM, 0.046 μM, 0.047 μM, 0.048 μM, 0.049 μM, 0.05 μM, 0.051 μM, 0.052 μM, 0.053 μM, 0.054 μM, 0.055 μM, 0.056 μM, 0.057 μM, 0.058 μM, 0.059 μM, 0.06 μM, 0.061 μM, 0.062 μM, 0.063 μM, 0.064 μM, 0.065 μM, 0.066 μM, 0.067 μM, 0.068 μM, 0.069 μM, 0.07 μM, 0.071 μM, 0.072 μM, 0.073 μM, 0.074 μM, 0.075 μM, 0.076 μM, 0.077 μM, 0.078 μM, 0.079 μM, 0.08 μM, 0.081 μM, 0.082 μM, 0.083 μM, 0.084 μM, 0.085 μM, 0.086 μM, 0.087 μM, 0.088 μM, 0.089 μM, 0.09 μM, 0.091 μM, 0.092 μM, 0.093 μM, 0.094 μM, 0.095 μM, 0.096 μM, 0.097 μM, 0.098 μM, 0.099 μM, 0.100 μM, 0.110 μM, 0.120 μM, 0.130 μM, 0.140 μM, 0.150 μM, 0.160 μM, 0.170 μM, 0.180 μM, 0.190 μM, 0.200 μM, 0.210 μM, 0.220 μM, 0.230 μM, 0.240 μM, 0.250 μM, 0.260 μM, 0.270 μM, 0.280 μM, 0.290 μM, 0.300 μM, 0.310 μM, 0.320 μM, 0.330 μM, 0.340 μM, 0.350 μM, 0.360 μM, 0.370 μM, 0.380 μM, 0.390 μM, 0.400 μM, 0.410 μM, 0.420 μM, 0.430 μM, 0.440 μM, 0.450 μM, 0.460 μM, 0.470 μM, 0.480 μM, 0.490 μM, 0.500 μM, 0.510 μM, 0.520 μM, 0.530 μM, 0.540 μM, 0.550 μM, 0.560 μM, 0.570 μM, 0.580 μM, 0.590 μM, 0.600 μM, 0.610 μM, 0.620 μM, 0.630 μM, 0.640 μM, 0.650 μM, 0.660 μM, 0.670 μM, 0.680 μM, 0.690 μM, 0.700 μM, 0.710 μM, 0.720 μM, 0.730 μM, 0.740 μM, 0.750 μM, 0.760 μM, 0.770 μM, 0.780 μM, 0.790 μM, 0.800 μM, 0.810 μM, 0.820 μM, 0.830 μM, 0.840 μM, 0.850 μM, 0.860 μM, 0.870 μM, 0.880 μM, 0.890 μM, 0.900 μM, 0.910 μM, 0.920 μM, 0.930 μM, 0.940 μM, 0.950 μM, 0.960 μM, 0.970 μM, 0.980 μM, 0.990 μM, 1.00 μM, 1.10 μM, 1.20 μM, 1.30 μM, 1.40 μM, 1.50 μM, 1.60 μM, 1.70 μM, 1.80 μM, 1.90 μM, 2.00 μM, 2.10 μM, 2.20 μM, 2.30 μM, 2.40 μM, 2.50 μM, 2.60 μM, 2.70 μM, 2.80 μM, 2.90 μM, 3.00 μM, 3.10 μM, 3.20 μM, 3.30 μM, 3.40 μM, 3.50 μM, 3.60 μM, 3.70 μM, 3.80 μM, 3.90 μM, 4.00 μM, 4.10 μM, 4.20 μM, 4.30 μM, 4.40 μM, 4.50 μM, 4.60 μM, 4.70 μM, 4.80 μM, 4.90 μM, 5.00 μM, 5.10 μM, 5.20 μM, 5.30 μM, 5.40 μM, 5.50 μM, 5.60 μM, 5.70 μM, 5.80 μM, 5.90 μM, 6.00 μM, 6.10 μM, 6.20 μM, 6.30 μM, 6.40 μM, 6.50 μM, 6.60 μM, 6.70 μM, 6.80 μM, 6.90 μM, 7.00 μM, 7.10 μM, 7.20 μM, 7.30 μM, 7.40 μM, 7.50 μM, 7.60 μM, 7.70 μM, 7.80 μM, 7.90 μM, 8.00 μM, 8.10 μM, 8.20 μM, 8.30 μM, 8.40 μM, 8.50 μM, 8.60 μM, 8.70 μM, 8.80 μM, 8.90 μM, 9.00 μM, 9.10 μM, 9.20 μM, 9.30 μM, 9.40 μM, 9.50 μM, 9.60 μM, 9.70 μM, 9.80 μM, 9.90 μM, or about 10.00 μM or any concentration therebetween.

As disclosed above, in some embodiments, a plurality of biomarkers can be analyzed in parallel for high-throughput and multiplex analysis. For example, the number of detection wells on a fluidic device may be any number between 1 and 200, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200. However, there may be fluidic devices that can have more than 200 detection wells. Each of the plurality of detection wells may be configured to detect a unique biomarker. In some cases, some of the wells may be configured to detect the same biomarker and used as replicates. In some cases, not all the biomarkers are used to diagnose the disease or condition; some may be used as a housekeeping biomarker to ensure the quality of the amplification reaction.

In some embodiments, the fluidic device is configured to detect at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, or more than 200 unique genes or biomarker genes. In some embodiments, the fluidic device is configured to detect a plurality of genes in a high-multiplex and/or high-throughput manner.

Each detection well may have a minimum amount of nucleic acid that is sufficient to detect the desired plurality of genes. In some embodiments, the amount of nucleic acid in each individual detection well is between about 0.1 ng to about 100 ng, e.g., about 0.1 ng, 0.2 ng, 0.3 ng, 0.4 ng, 0.5 ng, 0.6 ng, 0.7 ng, 0.8 ng, 0.9 ng, 1.0 ng, 2.0 ng, 3.0 ng, 4.0 ng, 5.0 ng, 6.0 ng, 7.0 ng, 8.0 ng, 9.0 ng, 10.0 ng, 11.0 ng, 12.0 ng, 13.0 ng, 14.0 ng, 15.0 ng, 16.0 ng, 17.0 ng, 18.0 ng, 19.0 ng, 20.0 ng, 21.0 ng, 22.0 ng, 23.0 ng, 24.0 ng, 25.0 ng, 26.0 ng, 27.0 ng, 28.0 ng, 29.0 ng, 30.0 ng, 31.0 ng, 32.0 ng, 33.0 ng, 34.0 ng, 35.0 ng, 36.0 ng, 37.0 ng, 38.0 ng, 39.0 ng, 40.0 ng, 41.0 ng, 42.0 ng, 43.0 ng, 44.0 ng, 45.0 ng, 46.0 ng, 47.0 ng, 48.0 ng, 49.0 ng, 50.0 ng, 51.0 ng, 52.0 ng, 53.0 ng, 54.0 ng, 55.0 ng, 56.0 ng, 57.0 ng, 58.0 ng, 59.0 ng, 60.0 ng, 61.0 ng, 62.0 ng, 63.0 ng, 64.0 ng, 65.0 ng, 66.0 ng, 67.0 ng, 68.0 ng, 69.0 ng, 70.0 ng, 71.0 ng, 72.0 ng, 73.0 ng, 74.0 ng, 75.0 ng, 76.0 ng, 77.0 ng, 78.0 ng, 78.0 ng, 79.0 ng, 80.0 ng, 81.0 ng, 82.0 ng, 83.0 ng, 84.0 ng, 85.0 ng, 86.0 ng, 87.0 ng, 88.0 ng, 89.0 ng, 90.0 ng, 91.0 ng, 92.0 ng, 93.0 ng, 94.0 ng, 95.0 ng, 96.0 ng, 97.0 ng, 98.0 ng, 99.0 ng, or about 100.0 ng, or any amount therebetween.

In relation to design of the fluidic device and processing the biological sample according step 508, the fluidic device can be structured to perform (e.g., optimized for) sample processing and characterization based upon a first set of informative biomarker components (i.e., informative genes), a second set of housekeeping biomarker components (e.g., housekeeping genes), and a third set of control components (e.g., control genes). Detection and analysis of the first set of informative biomarker components (i.e., informative genes) provides information regarding the condition of the subject (where exemplary conditions are described herein), the second set of housekeeping biomarker components (e.g., housekeeping genes) provides control from subject-to-subject, and the third set of control biomarker components (e.g., control genes) are indicative of proper functioning of the fluidic device (e.g. in relation to cartridge-to-cartridge consistency and reproducibility of analyses), with respect to proper sample preparation and proper amplification, respectively. The third set of control biomarker components is also used to normalize/provide corrections for characterizations (e.g., abundance characterizations) of other biomarker components.

In relation to the fluidic device 100 being structured for sample processing and characterization, as described, the selection of the first set of informative biomarkers, the second set of housekeeping biomarkers, and the third set of control biomarkers is based upon an assessment of biomarker targets that amplify suitably (e.g., using a LAMP assay), using the reaction chamber(s) and/or detection chamber(s) of the fluidic device 100, in coordination with instrument aspects described below. Upon processing the biological sample and analyzing signals related to the first set of informative biomarkers, the second set of housekeeping biomarkers, and the third set of control biomarkers, the system and method are structured to return a characterization of the biological sample based upon a composite biomarker.

According to a specific example, the first set of informative biomarkers includes the following genes: ANKRD22, ARG1, BATF, C3AR1, CD163, CEACAM1, CLEC5A, CTSL1, DEFA4, HERC5, HLA-DMB, IFI27, IFI44, IFI44L, IL18R1, IL1R2, ISG15, JUPv9, KCNJ2, LY86, OASL, OLFM4, PSMB9, RSAD2_PT4, S100A12_PT1, TDRD9_PT3, TGFBI, XAF1_PT4, and ZDHHC19

According to a specific example, the second set of housekeeping biomarkers includes the following genes: KPNA6, RREB1, and YWHAB.

According to a specific example, the third set of control biomarkers includes RNA controls.

As such, Step 508 can include processing the biological sample, in preparation for amplification and detection of a composite biomarker, where the composite biomarker is derived from expression levels of a combination of informative genes potentially represented in a sample: ANKRD22, ARG1, BATF, C3AR1, CD163, CEACAM1, CLECSA, CTSL1, DEFA4, HERC5, HLA-DMB, IFI27, IFI44, IFI44L, IL18R1, IL1R2, ISG15, JUPv9, KCNJ2, LY86, OASL, OLFM4, PSMB9, RSAD2_PT4, S100A12_PT1, TDRD9_PT3, TGFBI, XAF1_PT4, and ZDHHC19. However, variations of the method 500 can include other suitable informative genes or other loci of interest, for generation of a composite biomarker that is indicative of another condition.

In some embodiments, the method 500 can include a step 510 of measuring the expression level of a biomarker (e.g., generating signals indicative of an expression level of a composite biomarker) using the fluidic device disclosed herein and/or the instrument disclosed herein. In some embodiments, the step 510 may comprise acquiring an image of a detection well (and/or each detection well) in a detection chamber using an imaging system coupled to the instrument, as disclosed above. In some embodiments, the imaging system may acquire an image of the detection chamber to measure the fluorescence signal in one or more of the detection wells. In some embodiments, one or more images of the detection chamber may be processed by a software module coupled to the instrument 300, as disclosed above.

In some embodiments, the software module may be configured to quantify the fluorescence signal in the image of one or more detection wells. In some embodiments, the fluorescence intensity in the image may be correlated with the expression level (e.g., mRNA expression level) of a biomarker gene. In some embodiments, the fluorescence signal in the image may be processed to measure the mRNA expression level of a gene. In some embodiments, one or more biomarker genes may be a host immune response biomarker. In some embodiments, a biomarker gene may comprise a housekeeping gene. In some embodiments, the gene may comprise an internal control nucleic acid sequence. In some embodiments, the software module may normalize the expression level of a biomarker to the expression level of a control nucleic acid. In some embodiments, the software module may normalize the expression level of a biomarker to the expression level of a housekeeping gene. In some embodiments, a pre-processing unit of the software module may convert the fluorescence signal in an image of the detection chamber to a vector indicative of the expression level of a biomarker and feed the vector to the quantification unit. In some embodiments, the vector may comprise a plurality of quantitative features. The software module may be configured to detect the expression level of a plurality of genes or biomarker genes in a quantitative manner.

Generation of the composite biomarker in Step 510 can include measuring a respective expression level, from the fluidic device, of each of a set of biomarkers (e.g., genes, other loci of interest). In a specific example, the biological sample may potentially include one or more of a set of informative genes including: ANKRD22, ARG1, BATF, C3AR1, CD163, CEACAM1, CLECSA, CTSL1, DEFA4, HERC5, HLA-DMB, IFI27, IFI44, IFI44L, IL18R1, IL1R2, ISG15, JUPv9, KCNJ2, LY86, OASL, OLFM4, PSMB9, RSAD2_PT4, S100A12_PT1, TDRD9_PT3, TGFBI, XAF1_PT4, and ZDHHC19. Thus, Step 510 can include measuring the expression level of each informative gene, and generating a composite biomarker from the various expression levels of the respective genes, in order to return a characterization of a condition of a subject associated with the biological sample. The characterization can provide a categorization of the sample (e.g., viral infection, bacterial infection, sterile inflammation) and/or a severity of a condition, based upon composition of the composite biomarker (e.g., which genes are expressed, which genes are expressed above a threshold level) and/or other features of the composite biomarker (e.g., magnitude of the composite biomarker). However, variations of the method 500 can be adapted for other suitable informative genes or other loci of interest, for generation of a composite biomarker that is indicative of another condition.

Measuring can be performed in real-time during amplification (e.g., with a LAMP approach, with another amplification approach) at the fluidic device/cartridge, in order to monitor signals from the detection wells as the sample(s) is/are processed. Measuring in real-time can also be used to reduce sample processing time (e.g., if extent of amplification is suitable for returning an indication of a health condition associated with biomarker expression levels). Measuring can additionally or alternatively be performed at the end of the amplification process.

In some embodiments, a machine learning classifier in a software module may calculate a composite biomarker or a biomarker score (sometimes referred to just as a score) based on the vector that is useful in the prognosis and/or diagnosis of a disease or condition.

In some embodiments, the method 500 can include a step 512 of obtaining the results from the instrument and/or fluidic device (e.g., returning results characterizing a condition).

In some embodiments, it may take less than one (1) hour to complete the steps 504, 506, 508, 510, and 512 of the method 500. In some embodiments, it may take less than 30 min to complete the steps 504, 506, 508, 510, and 512 of the method 500. In some embodiments, it may take less than 15 min to complete the steps 504, 506, 508, 510, and 512 of the method 500. In some embodiments, it may take any time between about 0 min and about 60 min to complete the steps 504, 506, 508, 510, and 512 of the method 500, e.g., about 1 min, 2 min, 3 min, 4 min, 5 min, 6 min, 7 min, 8 min, 9 min, 10 min, 11 min, 12 min, 13 min, 14 min, 15 min, 16 min, 17 min, 18 min, 19 min, 20 min, 21 min, 22 min, 23 min, 24 min, 25 min, 26 min, 27 min, 28 min, 29 min, 30 min, 31 min, 32 min, 33 min, 34 min, 35 min, 36 min, 37 min, 38 min, 39 min, 40 min, 41 min, 42 min, 43 min, 44 min, 45 min, 46 min, 47 min, 48 min, 49 min, 50 min, 51 min, 52 min, 53 min, 54 min, 55 min, 56 min, 57 min, 58 min, 59 min, or about 60 min, any time therebetween. Methods provided herein may allow for the detection of a plurality of genes or biomarker genes in a rapid manner.

### 4. Example(s)

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### 4.1 Example 1—Characterization of a Subject Using the Fluidic Device

In an example, a method for evaluating a subject for a status of a condition can include: loading a biological sample of the subject into a fluidic device, wherein the biological sample comprises a volume of whole blood; performing a set of processes within the fluidic device; and measuring an expression level of a composite biomarker derived from a set of subcomponents present in the biological sample, using the fluidic device, wherein the subject is identified as having a state of the condition based on the expression level of the composite biomarker, and wherein the method takes no more than 35 minutes.

A variation of the example includes: receiving a biological sample into a collection tube, wherein the biological sample comprises a volume of whole blood from a subject, and wherein the volume of the biological sample is less than 600 microliters; coupling the collection tube with a fluidic device; and measuring an expression level of a composite biomarker derived from a set of subcomponents present in the biological sample, upon a) combining material from the biological sample with a reagent comprising a set of primers within the fluidic device, b) dividing the biological sample and the reagent across a set of chambers of the fluidic device, c) performing a loop-mediated isothermal amplification (LAMP) reaction within the set of chambers of the fluidic device, d) monitoring a set of signals from the set of chambers, the set of signals indicative of amplification progression associated with the set of subcomponents; and e) stopping the LAMP reaction and returning a result indicative of the expression level of the composite biomarker when a subset of the set of signals exceeds a threshold level; wherein the subject is identified as belonging to a category (e.g., as having or not having an infection, as having a condition, as having a state of severity of a condition, etc.) based on the expression level of the composite biomarker, and wherein the method takes no more than a duration of time (e.g., 35 minutes, etc.) from receiving the biological sample to measuring the expression level.

In the example, the biological sample comprises a volume from 400-600 microliters, with a ratio of blood:PAXgene® of 2.5:6.9 by volume.

Exemplary process run on a fluidic device/cartridge: In an example, the fluidic device and instrument can perform the following steps within respective chambers, channels, and regions of the fluidic device:

The fluidic device and/or instrument performs sample metering, upon coupling of the sample container with the fluidic device, by pulling the sample (e.g., blood combined with PAXgene®) into the metering chamber until it triggers the capacitive sensor in the instrument.

Then, the fluidic device and/or instrument performs sample lysis, with addition of lysis buffer to the blood/PAXgene® solution from the reagent storage zone. Addition of lysis buffer facilitates freeing of RNA from PAXgene® and for performance of lysis. However, lysis may already be complete prior to addition of lysis buffer, due to the activity of PAXgene®.

Then, the fluidic device and/or instrument performs RNA binding to functionalized particles (e.g., beads), wherein, in the specific example, dried magnetic beads are re-hydrated (e.g., using water), and then driven, with an alcohol solution, into the blood/lysis buffer mixture for combination and mixing.

Then, the fluidic device and/or instrument performs washing with a wash buffer from the reagent storage zone, with retention of the functionalized particles and their respective captured components in position (using a magnetic field), while supernatant is sent to waste (e.g., outside of the fluidic device, to a waste chamber 207 located on the fluidic device, etc.).

Then, the fluidic device and/or instrument performs washing with ethanol from the reagent storage zone, with retention of the functionalized particles and their respective captured components in position (using a magnetic field), while supernatant is sent to waste (e.g., outside of the fluidic device, to a waste chamber 207 located on the fluidic device, etc.).

Then, the fluidic device and/or instrument performs elution of RNA, with water for elution. Elution can also be performed using water combined with betaine.

Then, the fluidic device and/or instrument performs rehydration and mixing of master mix for amplification (e.g., LAMP amplification, etc.), where the master mix was previously dried down (e.g., in multiple separate groups)

Then, the fluidic device and/or instrument performs partitioning and rehydration of primers, where a homogenous mixture of RNA and master mix is driven to reaction wells of the reaction chamber for partitioning, and each reaction well contains the dried primer set that rehydrates with the homogenous mixture of RNA and master mix.

Then, the fluidic device and/or instrument performs amplification (e.g., isothermal amplification), followed by detection of expression levels of genes or other loci of interest represented in the sample.

Finally, expression levels of informative genes/loci of interest are analyzed and used to generate a composite biomarker, for sample characterization.

### 4.2 Example 2—Diagnosis of a Subject Using the Fluidic Device

To diagnose a subject for viral infection, bacterial infection, and severity of illness, a sample of approximately 2.5 mL of whole blood is collected into a PAXgene Blood RNA Tube. The PAXgene Blood RNA tube contains approximately 6.9 mL of additive which stabilizes intracellular RNA. The PAXgene tube containing blood from a subject is loaded into a fluidic device disclosed herein. The fluidic device is inserted into the instrument and the biological sample is then processed by the fluidic device inside the instrument. In approximately 30 minutes, the instrument provides an output reporting the likelihood of viral infection, the likelihood of bacterial infection, and the severity of illness.

### 5. Conclusions

The FIGURES illustrate the architecture, functionality and operation of possible implementations of systems, methods and computer program products according to preferred embodiments, example configurations, and variations thereof. In this regard, each block in the flowchart or block diagrams may represent a module, segment, or portion of code, which comprises one or more executable instructions for implementing the specified logical function(s). It should also be noted that, in some alternative implementations, the functions noted in the block can occur out of the order noted in the FIGURES. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

It should be understood from the foregoing that, while particular implementations have been illustrated and described, various modifications may be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents. It is intended that the following claims define the scope of the invention and that methods and structures within the scope of these claims and their equivalents be covered thereby.

As a person skilled in the art will recognize from the previous detailed description and from the figures and claims, modifications and changes can be made to the preferred embodiments of the invention without departing from the scope of this invention defined in the following claims.

What is claimed is:

1. A method for evaluating a subject for an infection, the method comprising:
loading a biological sample of the subject into a fluidic device, wherein the biological sample comprises a volume of blood;
performing a set of processes within the fluidic device; and
measuring an expression level of a composite biomarker derived from a set of subcomponents present in the biological sample, using the fluidic device,
wherein the subject is identified as having or not having an infection based on the expression level of the composite biomarker, and
wherein the method takes no more than 35 minutes.

2. The method of claim 1, wherein the method omits performance of deoxyribonucleic acid (DNA) degradation involving a Deoxyribonuclease (DNase), and wherein the method omits amplification of genomic DNA.

3. The method of claim 1, wherein the biological sample comprises intracellular ribonucleic acids (RNAs), and wherein the set of processes comprises combining the biological sample with an intracellular RNA stabilization solution.

4. The method of claim 1, wherein biological reagents stored on the fluidic device are prepared by convection drying to ensure stability at room temperature, and wherein said biological reagents are used for the set of processes.

5. The method of claim 1, wherein the set of processes comprises performing fluid agitation and homogenization of functionalized particles, with bubble mixing, within the fluidic device, thereby producing rapid binding of RNAs of the biological sample to capture molecules within the fluidic device.

6. The method of claim 1, wherein the volume of blood is less than 500 microliters, and wherein the set of subcomponents comprises a set of informative genes potentially represented in the biological sample.

7. The method of claim 6, wherein the set of informative genes comprises: ANKRD22, ARG1, BATF, C3AR1, CD163, CEACAM1, CLECSA, CTSL1, DEFA4, HERC5, HLA-DMB, IFI27, IFI44, IFI44L, IL18R1, IL1R2, ISG15, JUPv9, KCNJ2, LY86, OASL, OLFM4, PSMB9, RSAD2_PT4, S100A12_PT1, TDRD9_PT3, TGFBI, XAF1_PT4, and ZDHHC19.

8. The method of claim 7, wherein the composite biomarker is derived from expression levels of the set of informative genes.

9. The method of claim 1, wherein loading the biological sample comprises receiving the biological sample into a collection tube and coupling the collection tube with the fluidic device, wherein the step of loading the biological sample omits opening of the collection tube and omits manual transfer of the biological sample from the collection tube into the fluidic device.

10. The method of claim 1, wherein the fluidic device comprises a valve provided in a normally-closed operation mode, the valve structured for controlling flow of the biological sample into the fluidic device, and the method comprising providing increased resistance to opening the valve by:
providing the valve with a valve seat surrounded by a recess for an elastomeric membrane,
wherein the valve seat is one of: a) flush with and b) raised beyond the elastomeric membrane.

11. The method of claim 10, further comprising providing increased resistance to opening the valve with the collection tube by laser welding the elastomeric membrane to the valve seat.

12. The method of claim 10, further comprising providing increased resistance to opening the valve with the collection tube by providing a laser weld at one side of the valve seat, wherein the valve stays in the normally closed operation mode until a cracking pressure is exceeded upon coupling the collection tube with the fluidic device.

13. A method for evaluating a subject for an infection, the method comprising:
receiving a biological sample into a collection tube, wherein the biological sample comprises whole blood from a subject, and wherein the volume of the biological sample is less than 600 microliters;
coupling the collection tube with a fluidic device; and
measuring an expression level of a composite biomarker derived from a set of subcomponents present in the biological sample, upon combining material from the biological sample with a reagent comprising a set of primers within the fluidic device, dividing the biological sample and the reagent across a set of chambers of the fluidic device, performing a loop-mediated isothermal amplification (LAMP) reaction within the set of chambers of the fluidic device,
monitoring a set of signals from the set of chambers, the set of signals indicative of amplification progression associated with the set of subcomponents;
stopping the LAMP reaction and returning a result indicative of the expression level of the composite biomarker when a subset of the set of signals exceeds a threshold level; and
wherein the subject is identified as having or not having an infection based on the expression level of the composite biomarker, and
wherein the method takes no more than 35 minutes from receiving the biological sample to measuring the expression level.

14. The method of claim 13, wherein the method omits performance of deoxyribonucleic acid (DNA) degradation involving a Deoxyribonuclease (DNase), and wherein the method omits amplification of genomic DNA.

15. The method of claim 13, wherein biological reagents stored on the fluidic device are prepared by convection drying to ensure stability at room temperature, and wherein said biological reagents are used for the set of processes.

16. The method of claim 13, wherein the biological sample comprises intracellular ribonucleic acids (RNAs) resulting from lysing cells of the biological sample, and wherein the set of processes comprises combining the biological sample with an intracellular RNA stabilization solution.

17. The method of claim 13, wherein the set of subcomponents comprises a set of informative genes potentially represented in the biological sample, wherein the set of informative genes comprises: ANKRD22, ARG1, BATF, C3AR1, CD163, CEACAM1, CLEC5A, CTSL1, DEFA4, HERC5, HLA-DMB, IFI27, IFI44, IFI44L, IL18R1, IL1R2, ISG15, JUPv9, KCNJ2, LY86, OASL, OLFM4, PSMB9, RSAD2_PT4, S100A12_PT1, TDRD9_PT3, TGFBI, XAF1_PT4, and ZDHHC19.

18. The method of claim 13, further comprising detecting expression levels of a set of housekeeping genes and a set of control genes, and returning an indication of subject-to-subject control and proper functioning of the fluidic device based upon the expression levels of the set of housekeeping genes and the set of control genes.

19. The method of claim 13, wherein the fluidic device comprises a valve provided in a normally-closed operation mode, the method comprising providing increased resistance to opening the valve with the collection tube by:
providing the valve with a valve seat surrounded by a recess for an elastomeric membrane, wherein the valve seat is one of: a) flush with and b) raised beyond the elastomeric membrane,
laser welding the elastomeric membrane to the valve seat,
providing a laser weld at one side of the valve seat, wherein the valve stays in the normally closed operation mode until a cracking pressure is exceeded upon coupling the collection tube with the fluidic device.

20. The method of claim 19, wherein the cracking pressure is greater than 10 kPa.
